(19) United States
(12) Patent Application Publication
(10) Pub. No.: US 2023/0337989 A1
(43) Pub. Date: Oct. 26, 2023

(54) MOTION DATA DISPLAY METHOD AND SYSTEM

(71) Applicant: Shenzhen Shokz Co., Ltd., Shenzhen (CN)

(72) Inventors: Lei SU, Shenzhen (CN); Xin ZHOU, Shenzhen (CN); Meiqi LI, Shenzhen (CN); Fengyun LIAO, Shenzhen (CN)

(73) Assignee: Shenzhen Shokz Co., Ltd., Shenzhen (CN)

(21) Appl. No.: 18/200,777

(22) Filed: May 23, 2023

Related U.S. Application Data
(63) Continuation of application No. PCT/CN2021/093302, filed on May 12, 2021.

(51) Int. Cl. A61B 5/389; A61B 5/00

(52) U.S. Cl. CPC A61B 5/744; A61B 5/389; A61B 5/743

(57) ABSTRACT

A motion data display method and system is provided. The motion data and the reference motion data corresponding to the motion data of the user during moving of the user may be obtained; and the motion data of the user and the reference motion data may be combined with the virtual character, and the comparison between the motion data of the user and the reference motion data is intuitively displayed by using the animation of the virtual character, so that the user may intuitively find a difference between the motion data of the user and the reference motion data by observing the animation of the virtual character, so as to correct the motion during moving and move properly.

### RELATED APPLICATIONS

This application is a continuation application of PCT application No. PCT/CN2021/093302, filed on May 12, 2021, and the content of which is incorporated herein by reference in its entirety.

### TECHNICAL FIELD

This disclosure relates to the field of wearable device technologies, and in particular, to a motion data display method and system.

### BACKGROUND

As people are concerned about exercises and physical health, motion monitoring devices are booming. Currently, a motion monitoring device mainly monitors some physiological parameter information (for example, heart rate, body temperature, stride frequency, blood oxygen, etc.) during the movement of a user, displays physiological data to the user, and provides a suggestion based on the physiological data. However, in an actual scenario, sometimes there is a great difference between the physiological data and suggestion provided by the system and a sensation of the user. In the case where a user disagrees with system results and cannot intuitively understand an actual situation represented by the physiological data, the user’s trust in the device may be reduced consequently.

Therefore, a motion data display method and system capable of intuitively displaying physiological data of a user need to be provided.

### SUMMARY

This disclosure provides a motion data display method and system capable of intuitively displaying physiological data of a user.

In a first aspect, the present disclosure provides a motion data display method, including: obtaining motion data corresponding to motion signals at a plurality of measurement positions on a body of a user; obtaining reference motion data corresponding to the motion data; generating a model matching the user as a virtual character; and displaying the virtual character on a user interface to show a comparison between the motion data and the reference motion data.

In a second aspect, the present disclosure provides a motion data display system, including: at least one storage medium storing a set of instructions for motion data display; and at least one processor in communication with the at least one storage medium, where during operation, the at least one processor executes the set of instructions to: obtain motion data corresponding to motion signals at a plurality of measurement positions on a body of a user, obtain reference motion data corresponding to the motion data, generat a model matching the user as a virtual character, and display the virtual character on a user interface to show a comparison between the motion data and the reference motion data.

As can be seen from the foregoing technical solutions, for the motion data display method and system provided in this disclosure, the motion data (that is, physiological data) and the reference motion data corresponding to the motion data of the user during moving of the user may be obtained; and the motion data of the user and the reference motion data may be combined with the virtual character, and the comparison between the motion data of the user and the reference motion data is intuitively displayed by using the animation of the virtual character, so that the user may intuitively find a difference between the motion data of the user and the reference motion data by observing the animation of the virtual character, so as to correct the motion during moving and move properly.

### BRIEF DESCRIPTION OF THE DRAWINGS

This disclosure will be further described with some exemplary embodiments. The exemplary embodiments will be described in detail with reference to accompanying drawings. The embodiments are non-restrictive. In the embodiments, same numbers represent same structures.

FIG. 1 is a schematic diagram of an application scenario of a motion monitoring system according to some exemplary embodiments of this disclosure;

FIG. 2 is an exemplary schematic diagram of hardware and/or software of a wearable device according to some exemplary embodiments of this disclosure;

FIG. 3 is an exemplary schematic diagram of hardware and/or software of a computing device according to some exemplary embodiments of this disclosure;

FIG. 4 is an exemplary structural diagram of a wearable device according to some exemplary embodiments of this disclosure;

FIG. 5 is an exemplary flowchart of a motion monitoring method according to some exemplary embodiments of this disclosure;

FIG. 6 is an exemplary flowchart for monitoring a motion of a moving user according to some exemplary embodiments of this disclosure;

FIG. 7 is an exemplary flowchart for motion signal segmentation according to some exemplary embodiments of this disclosure;

FIG. 8 is an exemplary diagram of a normalization result of motion signal segmentation according to some exemplary embodiments of this disclosure;

FIG. 9 is an exemplary flowchart for myoelectric signal preprocessing according to some exemplary embodiments of this disclosure;

FIG. 10 is an exemplary flowchart for removing a burr signal according to some exemplary embodiments of this disclosure;

FIG. 11 is an exemplary flowchart for determining feature information corresponding to a posture signal according to some exemplary embodiments of this disclosure;

FIG. 12 is an exemplary flowchart for determining relative motion between different moving parts of a user according to some exemplary embodiments of this disclosure;

FIG. 13 is an exemplary flowchart for determining a conversion relationship between an original coordinate system and a specific coordinate system according to some exemplary embodiments of this disclosure;

FIG. 14 is an exemplary flowchart for determining a conversion relationship between an original coordinate system and a target coordinate system according to some exemplary embodiments of this disclosure;

FIG. 15A is an exemplary vector coordinate diagram of Euler angle data in an original coordinate system at a position of a forearm of a human body according to some exemplary embodiments of this disclosure;

FIG. 15B is an exemplary vector coordinate diagram of Euler angle data in an original coordinate system at another position of a forearm of a human body according to some exemplary embodiments of this disclosure;

FIG. 16A is an exemplary vector coordinate diagram of Euler angle data in a target coordinate system at a position of a forearm of a human body according to some exemplary embodiments of this disclosure;

FIG. 16B is an exemplary vector coordinate diagram of Euler angle data in a target coordinate system at another position of a forearm of a human body according to some exemplary embodiments of this disclosure;

FIG. 17 is an exemplary vector coordinate diagram of Euler angle data in a target coordinate system with a plurality of sensors according to some exemplary embodiments of this disclosure;

FIG. 18A is an exemplary result diagram of an original angular velocity according to some exemplary embodiments of this disclosure;

FIG. 18B is an exemplary result diagram of an angular velocity after filtering processing according to some exemplary embodiments of this disclosure;

FIG. 19 is an exemplary flowchart of a motion monitoring and feedback method according to some exemplary embodiments of this disclosure;

FIG. 20 is an exemplary flowchart of a model training disclosure according to some exemplary embodiments of this disclosure;

FIG. 21 is an exemplary flowchart of a motion data display method according to some exemplary embodiments of this disclosure;

FIG. 22 is an exemplary flowchart for generating a myoelectric animation according to some exemplary embodiments of this disclosure;

FIG. 23 is an exemplary flowchart for separately displaying myoelectric animations according to some exemplary embodiments of this disclosure;

FIG. 24A is a curve of myoelectric data of a biceps brachii over time according to some exemplary embodiments of this disclosure;

FIG. 24B is a curve of reference myoelectric data of a biceps brachii over time according to some exemplary embodiments of this disclosure;

FIG. 24C is a curve of myoelectric data of a biceps brachii over time after a linear duration adjustment according to some exemplary embodiments of this disclosure;

FIG. 24D is a curve of myoelectric data of a biceps brachii over time after a nonlinear duration adjustment according to some exemplary embodiments of this disclosure;

FIG. 25A is a schematic diagram of a user interface before a motion is started according to some exemplary embodiments of this disclosure;

FIG. 25B is a schematic diagram of a user interface in a motion process according to some exemplary embodiments of this disclosure;

FIG. 25C is a schematic diagram of a user interface after a motion is ended and before a next motion is started according to some exemplary embodiments of this disclosure;

FIG. 26 is an exemplary flowchart for generating a myoelectric animation according to some exemplary embodiments of this disclosure;

FIG. 27 is a schematic diagram of a user interface according to some exemplary embodiments of this disclosure;

FIG. 28 is an exemplary flowchart for generating a posture animation according to some exemplary embodiments of this disclosure; and

FIG. 29 is an exemplary flowchart for displaying a posture animation according to some exemplary embodiments of this disclosure.

### DETAILED DESCRIPTION

To clearly describe the technical solutions in the embodiments of this disclosure, the following briefly describes the accompanying drawings required for describing the embodiments. Apparently, the accompanying drawings in the following description merely show some examples or embodiments of this disclosure, and a person of ordinary skill in the art may further apply this disclosure to other similar scenarios based on the accompanying drawings without creative efforts. Unless apparently seen from contexts or otherwise specified, same reference numbers in the figures represent same structures or operations.

It should be understood that the terms “system”, “apparatus”, “unit”, and/or “module” used in this disclosure are/is for distinguishing between different components, elements, parts, portions, or assemblies at different levels. However, if other terms can achieve the same objective, the terms may be replaced with other expressions.

As shown in this disclosure or claims, unless explicitly indicated in an exceptional case in a context, the terms “a”, “one”, “one type of”, and/or “the” do not specially refer to singular forms, but may also include plural forms. Generally, the terms “comprise” and “include” merely indicate that explicitly identified steps and elements are included, but the steps and elements do not constitute an exclusive list, and a method or device may also include other steps or elements.

In this disclosure, a flowchart is used to describe operations performed by a system according to some exemplary embodiments of this disclosure. It should be understood that previous or subsequent operations are not necessarily performed in a precise order. Conversely, steps may be performed in a reverse order or processed simultaneously. In addition, other operations may also be added to the processes, or one operation or several operations may be removed from the processes.

This disclosure provides a motion monitoring system. The motion monitoring system may obtain a motion signal of a moving user, where the motion signal includes at least a myoelectric signal, a posture signal, an electrocardio signal, a respiratory frequency signal, or the like. The system may monitor the motion during moving of the user based on at least feature information corresponding to the myoelectric signal or feature information corresponding to the posture signal. For example, by using frequency information and amplitude information corresponding to the myoelectric signal, and an angular velocity, a direction of the angular velocity, an angular velocity value of the angular velocity, an angle, displacement information, stress, and the like corresponding to the posture signal, the system determines a motion type of the user, a quantity of motions, motion quality, and a motion time, or physiological parameter information or the like when the user performs the motion. In some exemplary embodiments, the motion monitoring system may further generate feedback about a bodybuilding motion of the user based on a result of analysis of the bodybuilding motion of the user, to guide the user in bodybuilding. For example, when the bodybuilding motion of the user is not up to standard, the motion monitoring system may send out prompt information (for example, a voice prompt, a vibration prompt, or a current stimulation) to the user. The motion monitoring system may be applied to a wearable device (for example, a garment, a wristband, or a helmet), a medical test device (for example, a myoelectric tester), a bodybuilding device, or the like. By obtaining the motion signal during moving of the user, the motion monitoring system may accurately monitor the motion of the user and provide feedback, without the involvement of professionals. Therefore, bodybuilding costs of the user can be reduced while bodybuilding efficiency of the user is improved.

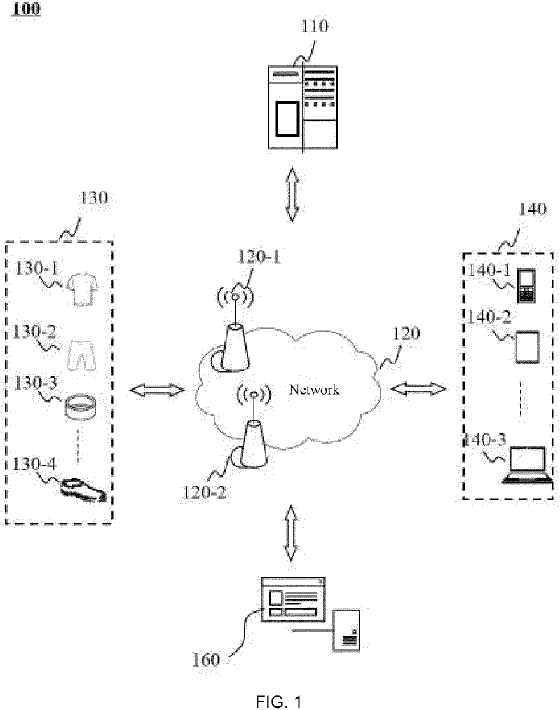

FIG. 1 is a schematic diagram of an application scenario of a motion monitoring system according to some exemplary embodiments of this disclosure. As shown in FIG. 1, the motion monitoring system 100 may include a processing device 110, a network 120, a wearable device 130, and a mobile terminal device 140. The motion monitoring system 100 may obtain a motion signal (for example, a myoelectric signal, a posture signal, an electrocardio signal, or a respiratory frequency signal) used to represent a motion during moving of a user, monitor the motion during moving of the user based on the motion signal of the user, and provide feedback.

For example, the motion monitoring system 100 may monitor a bodybuilding motion of the user and provide feedback. When the user wearing the wearable device 130 performs bodybuilding motion, the wearable device 130 may obtain the motion signal of the user. The processing device 110 or the mobile terminal device 140 may receive and analyze the motion signal of the user, to determine whether the bodybuilding motion of the user is up to standard and monitor the motion of the user. Specifically, monitoring the motion of the user may include determining a motion type of the motion, a quantity of motions, motion quality, and a motion time, or physiological parameter information or the like when the user performs the motion. Further, the motion monitoring system 100 may generate feedback about the bodybuilding motion of the user based on a result of analysis of the bodybuilding motion of the user, to guide the user in bodybuilding.

For example, the motion monitoring system 100 may monitor a running motion of the user and provide feedback. For example, when the user wearing the wearable device 130 performs running exercise, the motion monitoring system 100 may monitor whether the running motion of the user is up to standard, and whether a running time conforms to a health standard. When the running time of the user is too long, or the running motion is incorrect, the bodybuilding device may feedback a moving status of the user to the user, to prompt that the user needs to adjust the running motion or the running time.

In some exemplary embodiments, the processing device 110 may be configured to process information and/or data related to moving of the user. For example, the processing device 110 may receive the motion signal of the user (for example, the myoelectric signal, the posture signal, the electrocardio signal, or the respiratory frequency signal), and further extract feature information corresponding to the motion signal (for example, feature information corresponding to the myoelectric signal in the motion signal, or feature information corresponding to the posture signal). In some exemplary embodiments, the processing device 110 may perform specific signal processing, for example, signal segmentation or signal preprocessing (for example, signal correction processing or filtering processing), on the myoelectric signal or the posture signal captured by the wearable device 130. In some exemplary embodiments, the processing device 110 may also determine, based on the motion signal of the user, whether the motion of the user is correct. For example, the processing device 110 may determine, based on the feature information (for example, amplitude information or frequency information) corresponding to the myoelectric signal, whether the motion of the user is correct. As another example, the processing device 110 may determine, based on the feature information (for example, an angular velocity, a direction of the angular velocity, an acceleration of the angular velocity, an angle, displacement information, and stress) corresponding to the posture signal, whether the motion of the user is correct. As another example, the processing device 110 may determine, based on the feature information corresponding to the myoelectric signal and the feature information corresponding to the posture signal, whether the motion of the user is correct. In some exemplary embodiments, the processing device 110 may further determine whether the physiological parameter information during moving of the user conforms to the health standard. In some exemplary embodiments, the processing device 110 may further send a corresponding instruction for feeding back the moving status of the user. For example, when the user performs running exercise, the motion monitoring system 100 detects that the user has run for a too long time. In this case, the processing device 110 may send an instruction to the mobile terminal device 140 to prompt the user to adjust the running time. It should be noted that the feature information corresponding to the posture signal is not limited to the aforementioned angular velocity, direction of the angular velocity, acceleration of the angular velocity, angle, displacement information, stress, and the like, and may also be other feature information. Any parameter information that may reflect relative motion of the body of the user may be the feature information corresponding to the posture signal. For example, when a posture sensor is a strain sensor, a bending angle and a bending direction at the user’s joint may be obtained by measuring the resistance by the strain sensor, which varies with a tensile length.

In some exemplary embodiments, the processing device 110 may be a local or remote device. For example, through the network 120, the processing device 110 may access information and/or data stored in the wearable device 130 and/or the mobile terminal device 140. In some exemplary embodiments, the processing device 110 may be directly connected with the wearable device 130 and/or the mobile terminal device 140 to access information and/or data stored therein. For example, the processing device 110 may be located in the wearable device 130, and may exchange information with the mobile terminal device 140 through the network 120. As another example, the processing device 110 may be located in the mobile terminal device 140, and may exchange information with the wearable device 130 through the network. In some exemplary embodiments, the processing device 110 is executable on a cloud platform.

In some exemplary embodiments, the processing device 110 may process data and/or information related to motion monitoring to perform one or more of functions described in this disclosure. In some exemplary embodiments, the processing device 110 may obtain the motion signal captured by the wearable device 130 during moving of the user. In some exemplary embodiments, the processing device 110 may send a control instruction to the wearable device 130 or the mobile terminal device 140. The control instruction may control a switch status of the wearable device 130 and each sensor of the wearable device 130, and may further control the mobile terminal device 140 to send out prompt information. In some exemplary embodiments, the processing device 110 may include one or more sub processing devices (for example, single-core processing devices or multi-core processing devices).

The network 120 may facilitate the exchange of data and/or information in the motion monitoring system 100. In some exemplary embodiments, one or more components of the motion monitoring system 100 may send data and/or information to other components of the motion monitoring system 100 through the network 120. For example, the motion signal captured by the wearable device 130 may be transmitted to the processing device 110 through the network 120. As another example, a confirmation result about the motion signal in the processing device 110 may be transmitted to the mobile terminal device 140 through the network 120. In some exemplary embodiments, the network 120 may be any type of wired or wireless network.

The wearable device 130 refers to a garment or device that can be worn. In some exemplary embodiments, the wearable device 130 may include but is not limited to an upper garment apparatus 130-1, a pant apparatus 130-2, a wristband apparatus 130-3, a show apparatus 130-4, and the like. In some exemplary embodiments, the wearable device 130 may include a plurality of sensors. The sensors may obtain various motion signals (for example, the myoelectric signal, the posture signal, temperature information, a heart rate, and the electrocardio signal) during moving of the user. In some exemplary embodiments, the sensor may include but is not limited to one or more of a myoelectric sensor, a posture sensor, a temperature sensor, a humidity sensor, an electrocardio sensor, a blood oxygen saturation sensor, a Hall sensor, a galvanic skin sensor, a rotation sensor, and the like. For example, the myoelectric sensor may be disposed at a position of a muscle (for example, a biceps brachii, a triceps brachii, a latissimus dorsi, or a trapezius) of the human body in the upper garment apparatus 130-1, and the myoelectric sensor may fit onto the user’s skin and capture the myoelectric signal during moving of the user. As another example, an electrocardio sensor may be disposed near a left pectoral muscle of the human body in the upper garment apparatus 130-1, and the electrocardio sensor may capture the electrocardio signal of the user. As another example, the posture sensor may be disposed at a position of a muscle (for example, a gluteus maximus, a vastus lateralis, a vastus medialis, or a gastrocnemius) of the human body in the pant apparatus 130-2, and the posture sensor may capture the posture signal of the user. In some exemplary embodiments, the wearable device 130 may further provide feedback about the motion of the user. For example, during moving of the user, when a motion of a part of the body does not conform to the standard, a myoelectric sensor corresponding to the part may generate a stimulation signal (for example, a current stimulation or striking signal) to alert the user.

It should be noted that the wearable device 130 is not limited to the upper garment apparatus 130-1, the pant apparatus 130-2, the wristband apparatus 130-3, and the shoe apparatus 130-4 shown in FIG. 1, but may further include any other device that may perform motion monitoring, such as a helmet apparatus or a kneepad apparatus. This is not limited herein. Any device that can use the motion monitoring method included in this disclosure shall fall within the protection scope of this disclosure.

In some exemplary embodiments, the mobile terminal device 140 may obtain information or data from the motion monitoring system 100. In some exemplary embodiments, the mobile terminal device 140 may receive processed motion data from the processing device 110, and then feedback a motion record or the like based on the processed motion data. Exemplary feedback manners may include but are not limited to a voice prompt, an image prompt, a video presentation, a text prompt, and the like. In some exemplary embodiments, the user may obtain a motion record during moving of the user by using the mobile terminal device 140. For example, the mobile terminal device 140 may be connected with the wearable device 130 through the network 120 (for example, a wired connection or a wireless connection), and the user may obtain the motion record during moving of the user by using the mobile terminal device 140, where the motion record may be transmitted to the processing device 110 through the mobile terminal device 140. In some exemplary embodiments, the mobile terminal device 140 may include one or any combination of a mobile apparatus 140-1, a tablet computer 140-2, a notebook computer 140-3, and the like. In some exemplary embodiments, the mobile apparatus 140-1 may include a mobile phone, a smart home apparatus, a smart mobile apparatus, a virtual reality apparatus, an augmented reality apparatus, or the like, or any combination thereof. In some exemplary embodiments, the smart home apparatus may include a smart appliance control apparatus, a smart monitoring apparatus, a smart television, a smart camera, or the like, or any combination thereof. In some exemplary embodiments, the smart mobile apparatus may include a smartphone, a personal digital assistant (PDA), a game apparatus, a navigation apparatus, a point of sale (POS) apparatus, or the like, or any combination thereof. In some exemplary embodiments, the virtual reality apparatus and/or the augmented reality apparatus may include a virtual reality helmet, virtual reality glasses, virtual reality eye masks, an augmented reality helmet, augmented reality glasses, augmented reality eye masks, or the like, or any combination thereof.

In some exemplary embodiments, the motion monitoring system 100 may further include a motion data display system 160. The motion data display system 160 may be configured to process information and/or data related to the motion of the user, and combine the information and/or data with a virtual character for visual displaying on a user interface of the mobile terminal device 140, to facilitate viewing by the user. For example, the motion data display system 160 may receive motion data of the user, for example, the motion signal such as the myoelectric signal, the posture signal, the electrocardio signal, or the respiratory frequency signal, or as another example, the feature information (for example, the feature information corresponding to the myoelectric signal in the motion signal or the feature information corresponding to the posture signal) obtained by the processing device 110 by performing feature processing on the motion signal, or as another example, a signal obtained by the processing device 110 by performing specific signal processing on the motion signal, such as signal segmentation or signal preprocessing (for example, signal correction processing or filtering processing). The motion data display system 160 may compare the motion data with reference motion data, and combine a comparison result with the virtual character to generate an animation of the virtual character and send the animation to the mobile terminal device 140 for displaying. The reference motion data will be described in detail in the subsequent description. For example, when the user performs a motion of biceps lifting, the motion data display system 160 may receive motion data of the user during biceps lifting, such as a myoelectric signal of the biceps brachii, a myoelectric signal of the trapezius, a motion posture of the forearm, or a motion posture of the upper arm. The motion data display system 160 may compare the motion data of the user with reference motion data of the biceps lifting motion stored in the motion monitoring system 100, and present a comparison result on the virtual character to form an animation of the virtual character for displaying. The user may clearly and visually find a difference between the motion data of the user and the reference motion data by using the animation of the virtual character, such as a difference in muscle force-generating positions, a force difference, or a motion posture difference, so as to adjust the motion during moving.

In some exemplary embodiments, the motion data display system 160 may be integrated with the processing device 110. In some exemplary embodiments, the motion data display system 160 may alternatively be integrated with the mobile terminal device 140. In some exemplary embodiments, the motion data display system 160 may alternatively exist independently of the processing device 110 and the mobile terminal device 140. The motion data display system 160 may be in communication with the processing device 110, the wearable device 130, and the mobile terminal device 140 to transmit and exchange information and/or data. In some exemplary embodiments, through the network 120, the motion data display system 160 may access information and/or data stored in the processing device 110, the wearable device 130, and/or the mobile terminal device 140. In some exemplary embodiments, the wearable device 130 may be directly connect with the processing device 110 and/or the mobile terminal device 140 to access information and/or data stored therein. For example, the motion data display system 160 may be located in the processing device 110, and may exchange information with the wearable device 130 and the mobile terminal device 140 through the network 120. As another example, the motion data display system 160 may be located in the mobile terminal device 140, and may exchange information with the processing device 110 and the wearable device 130 through the network. In some exemplary embodiments, the motion data display system 160 may be executable on the cloud platform, and may exchange information with the processing device 110, the wearable device 130, and the mobile terminal device 140 through the network.

For ease of presentation, the motion data display system 160 located in the mobile terminal device 140 is hereinafter used as an example in the following description.

In some exemplary embodiments, the motion data display system 160 may process data and/or information related to motion data to perform one or more of functions described in this disclosure. In some exemplary embodiments, the motion data display system 160 may obtain the motion data during moving of the user, for example, the motion signal captured by the wearable device 130 during moving of the user, or as another example, data obtained after the motion signal captured by the wearable device 130 during moving of the user may be processed by the processing device 110. In some exemplary embodiments, the motion data display system 160 may send a control instruction to the mobile terminal device 140 to control displaying of the user interface of the mobile terminal device 140.

In some exemplary embodiments, the motion monitoring system 100 may further include a database. The database may store data (for example, an initially set threshold condition) and/or an instruction (for example, a feedback instruction). In some exemplary embodiments, the database may store data obtained from the wearable device 130 and/or the mobile terminal device 140. In some exemplary embodiments, the database may store information and/or instructions for execution or use by the processing device 110, to perform the exemplary method described in this disclosure. In some exemplary embodiments, the database may be connected to the network 120 to communicate with one or more components of the motion monitoring system 100 (for example, the processing device 110, the wearable device 130, and the mobile terminal device 140). Through the network 120, the one or more components of the motion monitoring system 100 may access data or instructions stored in the database. In some exemplary embodiments, the database may be directly connected with or communicate with the one or more components of the motion monitoring system 100. In some exemplary embodiments, the database may be a part of the processing device 110.

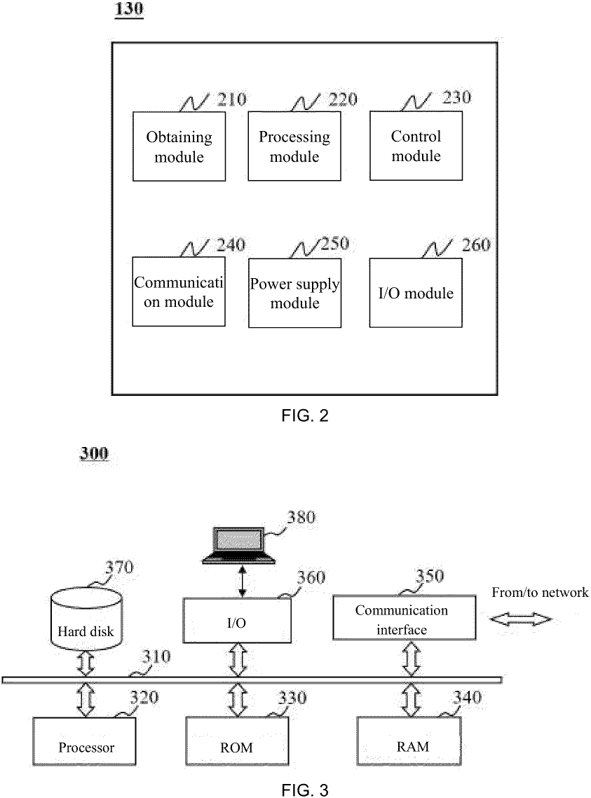

FIG. 2 is an exemplary schematic diagram of hardware and/or software of a wearable device 130 according to some exemplary embodiments of this disclosure. As shown in FIG. 2, the wearable device 130 may include an obtaining module 210, a processing module 220 (also referred to as a processor), a control module 230 (also referred to as a main controller, an MCU, or a controller), a communication module 240, a power supply module 250, and an input/output (I/O) module 260.

The obtaining module 210 may be configured to obtain a motion signal of a moving user. In some exemplary embodiments, the obtaining module 210 may include a sensor unit, where the sensor unit may be configured to obtain one or more motion signals during moving of the user. In some exemplary embodiments, the sensor unit may include but is not limited to one or more of a myoelectric sensor, a posture sensor, an electrocardio sensor, a respiration sensor, a temperature sensor, a humidity sensor, an inertial sensor, a blood oxygen saturation sensor, a Hall sensor, a galvanic skin sensor, a rotation sensor, and the like. In some exemplary embodiments, the motion signal may include one or more of a myoelectric signal, a posture signal, an electrocardio signal, a respiratory frequency, a temperature signal, a humidity sensor, and the like. The sensor unit may be placed in different positions of the wearable device 130 depending on a type of a motion signal to be obtained. For example, in some exemplary embodiments, the myoelectric sensor (also referred to as an electrode element) may be disposed at a position of a muscle of the human body, and the myoelectric sensor may be configured to capture a myoelectric signal during moving of the user. The myoelectric signal and feature information (for example, frequency information or amplitude information) corresponding to the myoelectric signal may reflect a status of the muscle during moving of the user. The posture sensor may be disposed at different positions of the human body (for example, positions corresponding to a torso, four limbs, and joints, in the wearable device 130), and the posture sensor may be configured to capture a posture signal during moving of the user. The posture signal and feature information (for example, a direction of an angular velocity, an angular velocity value, an acceleration value of the angular velocity, an angle, displacement information, and stress) corresponding to the posture signal may reflect a posture during moving of the user. The electrocardio sensor may be disposed at a position around a chest of the human body, and the electrocardio sensor may be configured to capture electrocardio data during moving of the user. The respiration sensor may be disposed at a position around the chest of the human body, and the respiration sensor may be configured to capture respiratory data (for example, a respiratory frequency and a respiratory amplitude) during moving of the user. The temperature sensor may be configured to capture temperature data (for example, a shell temperature) during moving of the user. The humidity sensor may be configured to capture humidity data of an external environment during moving of the user.

The processing module 220 may process data from the obtaining module 210, the control module 230, the communication module 240, the power supply module 250, and/or the input/output module 260. For example, the processing module 220 may process the motion signal during moving of the user from the obtaining module 210. In some exemplary embodiments, the processing module 220 may preprocess the motion signal (for example, the myoelectric signal or the posture signal) obtained by the obtaining module 210. For example, the processing module 220 performs segmentation processing on the myoelectric signal or the posture signal during moving of the user. As another example, the processing module 220 may perform preprocessing (for example, filtering processing or signal correction processing) on the myoelectric signal during moving of the user, to improve quality of the myoelectric signal. As another example, the processing module 220 may determine, based on the posture signal during moving of the user, feature information corresponding to the posture signal. In some exemplary embodiments, the processing module 220 may process an instruction or an operation from the input/output module 260. In some exemplary embodiments, processed data may be stored in a memory or a hard disk. In some exemplary embodiments, the processing module 220 may transmit, through the communication module 240 or a network 120, the data processed by the processing module 220 to one or more components of the motion monitoring system 100. For example, the processing module 220 may send a motion monitoring result of the user to the control module 230, and the control module 230 may execute a subsequent operation or instruction based on the motion monitoring result.

The control module 230 may be connected with other modules in the wearable device 130. In some exemplary embodiments, the control module 230 may control a running status of another module in the wearable device 130. For example, the control module 230 may control a power supply status (for example, a normal mode or a power saving mode), a power supply time, and the like of the power supply module 250. As another example, the control module 230 may control the input/output module 260 based on the motion determining result of the user, and may further control a mobile terminal device 140 to send a motion feedback result of the user to the user. When there is a problem with the motion (for example, the motion does not conform to a standard) during moving of the user, the control module 230 may control the input/output module 260, and may further control the mobile terminal device 140 to provide feedback for the user, so that the user may learn the moving status of the user and adjust the motion. In some exemplary embodiments, the control module 230 may further control one or more sensors in the obtaining module 210 or another module to provide feedback about the human body. For example, when the force of a muscle is excessive during moving of the user, the control module 230 may control an electrode module at a position of the muscle to electrically stimulate the user to prompt the user to adjust the motion in time.

In some exemplary embodiments, the communication module 240 may be configured to exchange information or data. In some exemplary embodiments, the communication module 240 may be used for communications between internal components of the wearable device 130. For example, the obtaining module 210 may send the motion signal (for example, the myoelectric signal or the posture signal) of the user to the communication module 240, and the communication module 240 may send the motion signal to the processing module 220. In some exemplary embodiments, the communication module 240 may be further used for communications between the wearable device 130 and other components of the motion monitoring system 100. For example, the communication module 240 may send status information (for example, a switch status) of the wearable device 130 to a processing device 110, and the processing device 110 may monitor the wearable device 130 based on the status information. The communication module 240 may use wired, wireless, or hybrid wired and wireless technologies.

In some exemplary embodiments, the power supply module 250 may supply power to other components of the motion monitoring system 100.

The input/output module 260 may obtain, transmit, and send signals. The input/output module 260 may be connected to or communicate with other components of the motion monitoring system 100. The other components of the motion monitoring system 100 may be connected or communicate through the input/output module 260.

It should be noted that the foregoing description of the motion monitoring system 100 and modules thereof is merely for ease of description and is not intended to limit one or more embodiments of this disclosure to the scope of the illustrated embodiments. It may be understood that, after learning the principle of the system, a person skilled in the art may arbitrarily combine the modules, or connect a constituent subsystem to other modules, or omit one or more modules, without departing the principle. For example, the obtaining module 210 and the processing module 220 may be one module, and the module may have functions for obtaining and processing the motion signal of the user. As another example, alternatively, the processing module 220 may not be disposed in the wearable device 130, but is integrated in the processing device 110. All such variations shall fall within the protection scope of one or more embodiments of this disclosure.

FIG. 3 is an exemplary schematic diagram of hardware and/or software of a computing device 300 according to some exemplary embodiments of this disclosure. In some exemplary embodiments, a processing device 110 and/or a mobile terminal device 140 may be implemented on the computing device 300. In some exemplary embodiments, a motion data display system 160 may be implemented on the computing device 300. As shown in FIG. 3, the computing device 300 may include an internal communications bus 310, a processor 320, a read-only memory 330, a random access memory 340, a communication port 350, an input/output interface 360, a hard disk 370, and a user interface 380.

The internal communication bus 310 may implement data communications between various components in the computing device 300. For example, the processor 320 may send data to other hardware such as the memory or the input/output port 360 through the internal communication bus 310.

The processor 320 may execute a computer instruction (program code), and perform a function of a motion monitoring system 100 described in this disclosure. The computer instruction may include a program, an object, a component, a data structure, a process, a module, and a function (the function is a particular function as described in this disclosure). For example, the processor 320 may process a motion signal (for example, a myoelectric signal or a posture signal) obtained from a wearable device 130 or/and a mobile terminal device 140 of the motion monitoring system 100 during moving of a user, and monitor a moving motion of the user based on the motion signal during moving of the user. For a purpose of description only, only one processor is depicted in the computing device 300 in FIG. 3. However, it should be noted that the computing device 300 in this disclosure may also include a plurality of processors.

The memory (for example, a read-only memory (ROM) 330, a random access memory (RAM) 340, or a hard disk 370) of the computing device 300 may store data/information obtained from any other component of the motion monitoring system 100. In some exemplary embodiments, the memory of the computing device 300 may be located in the wearable device 130, or may be located in the processing device 110.

The input/output interface 360 may be configured to input or output signals, data, or information. In some exemplary embodiments, the input/output interface 360 may allow the user to interact with the motion monitoring system 100.

The hard disk 370 may be configured to store information and data generated by the processing device 110 or received from the processing device 110. For example, the hard disk 370 may store user confirmation information of the user. In some exemplary embodiments, the hard disk 370 may be disposed in the processing device 110 or the wearable device 130. The user interface 380 may implement interaction and information exchange between the computing device 300 and the user. In some exemplary embodiments, the user interface 380 may be configured to present a motion record generated by the motion monitoring system 100 to the user. In some exemplary embodiments, the user interface 380 may include a physical display, such as a display with a speaker, an LCD display, an LED display, an OLED display, or an electronic ink (E-Ink) display.

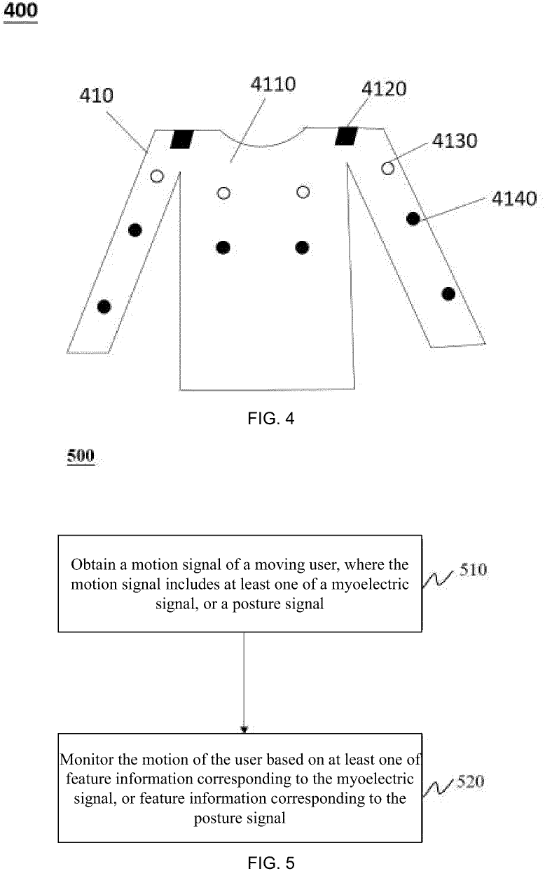

FIG. 4 is an exemplary structural diagram of a wearable device according to some exemplary embodiments of this disclosure. To further describe the wearable device, an upper garment is used herein for illustration. As shown in FIG. 4, a wearable device 400 may include an upper garment 410. The upper garment 410 may include an upper garment base 4110, at least one upper garment processing module 4120, at least one upper garment feedback module 4130, at least one upper garment obtaining module 4140, and the like. The upper garment base 4110 may be a garment worn on an upper part of the human body. In some exemplary embodiments, the upper garment base 4110 may include a short-sleeved T-shirt, a long-sleeved T-shirt, a shirt, a coat, or the like. The at least one upper garment processing module 4120 and the at least one upper garment obtaining module 4140 may be located in areas of the upper garment base 4110 that fit onto different parts of the human body. The at least one upper garment feedback module 4130 may be located in any position of the upper garment base 4110. The at least one upper garment feedback module 4130 may be configured to feedback moving status information of an upper part of the body of a user. Exemplary feedback manners may include but are not limited to a voice prompt, a text prompt, a pressure prompt, a current stimulation, and the like. In some exemplary embodiments, the at least one upper garment obtaining module 4140 may include but is not limited to one or more of a posture sensor, an electrocardio sensor, a myoelectric sensor, a temperature sensor, a humidity sensor, an inertial sensor, an acid-base sensor, an acoustic transducer, and the like. The sensor in the upper garment obtaining module 4140 may be placed in different positions of the body of the user based on different signals to be measured. For example, when the posture sensor is configured to obtain a posture signal during moving of the user, the posture sensor may be placed at positions corresponding to a torso, two arms, and joints of the human body, in the upper garment base 4110. As another example, when the myoelectric sensor is configured to obtain a myoelectric signal during moving of the user, the myoelectric sensor may be located near the user’s muscle to be measured. In some exemplary embodiments, the posture sensor may include but is not limited to an acceleration three-axis sensor, an angular velocity three-axis sensor, a magnetic sensor, or the like, or any combination thereof. For example, one posture sensor may include an acceleration three-axis sensor or an angular velocity three-axis sensor. In some exemplary embodiments, the posture sensor may further include a strain sensor. The strain sensor may be a sensor based on a strain generated due to forced deformation of an object to be measured. In some exemplary embodiments, the strain sensor may include but is not limited to one or more of a strain force sensor, a strain pressure sensor, a strain torque sensor, a strain displacement sensor, a strain acceleration sensor, and the like. For example, the strain sensor may be disposed at a joint position of the user, and a bending angle and a bending direction at the user’s joint may be obtained by measuring a magnitude of resistance that varies with a tensile length in the strain sensor. It should be noted that the upper garment 410 may further include other modules, for example, a power supply module, a communication module, and an input/output module, in addition to the upper garment base 4110, the upper garment processing module 4120, the upper garment feedback module 4130, and the upper garment obtaining module 4140 described above. The upper garment processing module 4120 may be similar to the processing module 220 in FIG. 2. The upper garment obtaining module 4140 may be similar to the obtaining module 210 in FIG. 2. For detailed descriptions about the modules in the upper garment 410, reference may be made to related descriptions in FIG. 2 of this disclosure. Details will not be described herein again.

FIG. 5 is an exemplary flowchart of a motion monitoring method according to some exemplary embodiments of this disclosure. As shown in FIG. 5, the process 500 may include the following steps.

Step 510: Obtain a motion signal of a moving user.

In some exemplary embodiments, step 510 may be performed by the obtaining module 210. The motion signal refers to body parameter information during moving of the user. In some exemplary embodiments, the body parameter information may include but is not limited to one or more of a myoelectric signal, a posture signal, an electrocardio signal, a temperature signal, a humidity sensor, a blood oxygen concentration, a respiratory frequency, and the like. In some exemplary embodiments, a myoelectric sensor in the obtaining module 210 may capture a myoelectric signal during moving of the user. For example, when the user performs seated chest fly, a myoelectric sensor corresponding to a position of a pectoral muscle, a latissimus dorsi, or the like of a human body, in a wearable device may capture a myoelectric signal at a corresponding muscle position of the user. As another example, when the user performs deep squat, a myoelectric sensor corresponding to a position of a gluteus maximus, a quadriceps femoris, or the like of the human body, in the wearable device may capture a myoelectric signal at a corresponding muscle position of the user. As another example, when the user performs running exercise, a myoelectric sensor corresponding to a position of a gastrocnemius or the like of the human body, in the wearable device may capture a myoelectric signal at the position of the gastrocnemius or the like of the human body. In some exemplary embodiments, a posture sensor in the obtaining module 210 may capture a posture signal during moving of the user. For example, when the user performs barbell horizontal-pushing motion, a posture sensor corresponding to a position of a triceps brachii or the like of the human body, in the wearable device may capture a posture signal at the position of the triceps brachii or the like of the user. As another example, when the user performs a dumbbell bird motion, a posture sensor disposed at a position of a deltoid or the like of the human body may capture a posture signal at the position of the deltoid or the like of the user. In some exemplary embodiments, the obtaining module 210 may include a plurality of posture sensors, and the plurality of posture sensors may obtain posture signals of a plurality of parts of the body during moving of the user, where the plurality of posture signals may reflect a status of relative moving between different parts of the human body. For example, a posture signal at an arm and a posture signal at a torso may reflect a moving status of the arm relative to the torso. In some exemplary embodiments, posture signals are associated with types of posture sensors. For example, when a posture sensor is an angular velocity three-axis sensor, an obtained posture signal may be angular velocity information. As another example, when posture sensors are an angular velocity three-axis sensor and an acceleration three-axis sensor, the obtained posture signals may be angular velocity information and acceleration information. As another example, when a posture sensor is a strain sensor, the strain sensor may be disposed at a joint position of the user, a posture signal obtained by measuring a magnitude of resistance varying with a tensile length in the strain sensor may be displacement information, stress, and the like, and the posture signal may represent a bending angle and a bending direction at the user’s joint. It should be noted that parameter information that can be used to reflect relative motion of the body of the user may all be feature information corresponding to posture signals, and different types of posture sensors may be used to obtain the information based on feature information types.

In some exemplary embodiments, the motion signal may include a myoelectric signal of a specific part of the body of the user and a posture signal of the specific part. The myoelectric signal and the posture signal may reflect a moving status of the specific part of the body of the user from different perspectives. In short, the posture signal of the specific part of the body of the user may reflect a motion type, a motion amplitude, a motion frequency, or the like of the specific part. The myoelectric signal may reflect a muscle status of the specific part during moving. In some exemplary embodiments, myoelectric signals and/or posture signals of the same part of the body may be used to better evaluate whether the motion of the part is up to standard.

Step 520: Monitor the motion of the user based on at least one of feature information corresponding to the myoelectric signal, or feature information corresponding to the posture signal.

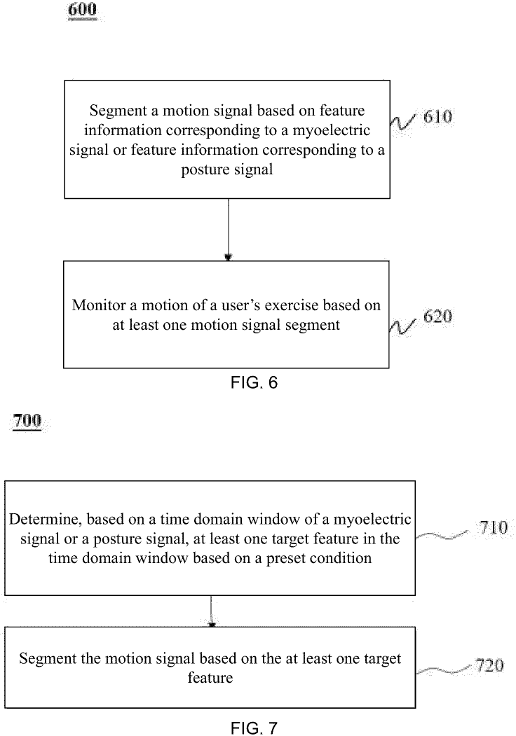

In some exemplary embodiments, this step may be performed by the processing module 220 and/or the processing device 110. In some exemplary embodiments, the feature information corresponding to the myoelectric signal may include but is not limited to one or more of frequency information, amplitude information, and the like. The feature information corresponding to the posture signal is parameter information used to represent relative motion of the body of the user. In some exemplary embodiments, the feature information corresponding to the posture signal may include but is not limited to one or more of a direction of an angular velocity, an angular velocity value, an acceleration value of the angular velocity, and the like. In some exemplary embodiments, the feature information corresponding to the posture signal may further include an angle, displacement information (for example, a tensile length in a strain sensor), stress, and the like. For example, when a posture sensor is a strain sensor, the strain sensor may be disposed at a joint position of the user, a posture signal obtained by measuring a magnitude of resistance varying with a tensile length in the strain sensor may be displacement information, stress, and the like, and the posture signal may represent a bending angle and a bending direction at the user’s joint. In some exemplary embodiments, the processing module 220 and/or the processing device 110 may extract the feature information (for example, the frequency information and the amplitude information) corresponding to the myoelectric signal or the feature information (for example, the direction of the angular velocity, the angular velocity value, the acceleration value of the angular velocity, the angle, the displacement information, stress, and the like) corresponding to the posture signal, and monitor the motion of the user based on the feature information corresponding to the myoelectric signal or the feature information corresponding to the posture signal. Herein, monitoring the motion during moving of the user includes monitoring information related to the motion of the user. In some exemplary embodiments, the information related to the motion may include one or more of a motion type of the user, a quantity of motions, motion quality (for example, whether the motion of the user conforms to a standard), a motion time, and the like. The motion type refers to a bodybuilding motion taken during moving of the user. In some exemplary embodiments, the motion type may include but is not limited to one or more of seated chest fly, deep squat exercise, hard pull exercise, plank exercise, running, swimming, and the like. The motion quantity is a quantity of motions performed during moving of the user. For example, the user performs seated chest fly for 10 times, and the motion quantity herein is 10. The motion quality refers to a degree of conformity of a bodybuilding motion performed by the user to a standard bodybuilding motion. For example, when the user performs deep squat, the processing device 110 may determine a motion type of the motion of the user based on feature information corresponding to a motion signal (myoelectric signal and posture signal) at a specific muscle position (gluteus maximus, quadriceps femoris, or the like), and determine motion quality of the deep squat motion of the user based on a motion signal of a standard deep squat motion. The motion time refers to the time corresponding to one or more motion types of the user or the total time of the motion process. For details about monitoring the motion during moving of the user based on the feature information corresponding to the myoelectric signal and/or the feature information corresponding to the posture signal, reference may be made to FIG. 6 of this disclosure and related descriptions thereof.

FIG. 6 is an exemplary flowchart for monitoring a motion during moving of a user according to some exemplary embodiments of this disclosure. As shown in FIG. 6, the process 600 may include the following steps.

Step 610: Segment a motion signal based on feature information corresponding to a myoelectric signal or feature information corresponding to a posture signal.

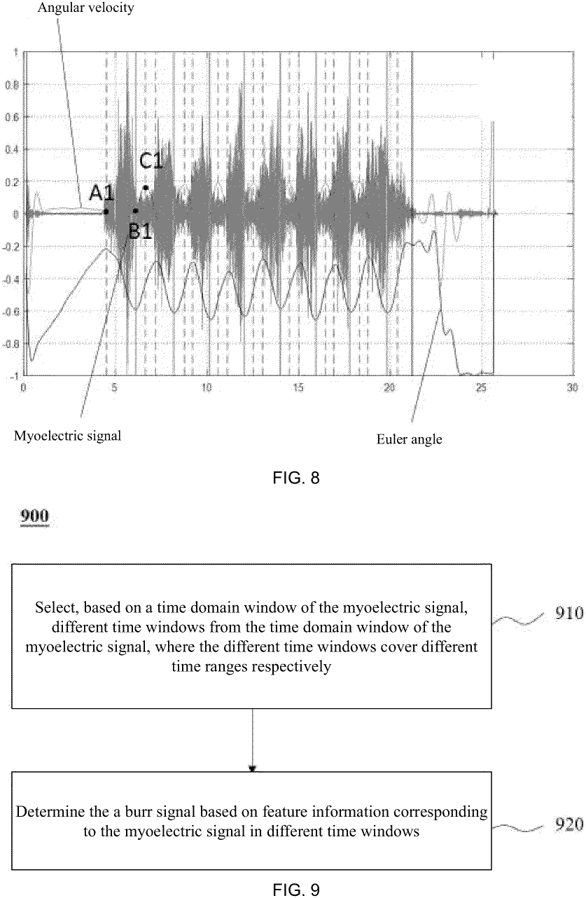

In some exemplary embodiments, this step may be performed by the processing module 220 and/or the processing device 110. A process of capturing a motion signal (for example, a myoelectric signal or a posture signal) during moving of a user is continuous, and a motion during moving of the user may be a combination of a plurality of groups of motions or a combination of motions of different motion types. To analyze various motions during moving of the user, the processing module 220 may segment the user motion signal based on the feature information corresponding to the myoelectric signal or the feature information corresponding to the posture signal during moving of the user. The motion signal segmentation herein means dividing the motion signal into signal segments of a same duration or different durations, or extracting one or more signal segments of a specific duration from the motion signal. In some exemplary embodiments, each motion signal segment may correspond to one or more full motions. For example, when the user takes deep squat exercise, squatting from a standing posture and then standing up to resume the standing posture by the user may be considered as completing a deep squat motion by the user, and a motion signal captured by the obtaining module 210 in this process may be considered as a motion signal segment (or period), and after that, a motion signal captured by the obtaining module 210 when the user completes a next squat motion is considered as another motion signal segment. In some exemplary embodiments, each motion signal segment may alternatively correspond to a partial motion of the user, and the partial motion herein may be understood as a partial motion in a full motion. For example, when the user takes deep squat exercise, squatting from a standing posture by the user may be considered as a motion segment, and then standing up to resume the standing posture may be considered as another motion segment. During moving of the user, a change in each motion step may cause a myoelectric signal and a posture signal of a corresponding part to change. For example, when the user performs deep squat, a fluctuation of a myoelectric signal and a posture signal at a corresponding muscle of a corresponding part of the body (for example, an arm, a leg, a hip, or an abdomen) when the user stands is small, and a fluctuation of the myoelectric signal and the posture signal at the corresponding muscle of the corresponding part of the body of the user when the user squats from a standing posture is large. For example, amplitude information corresponding to signals of different frequencies in the myoelectric signal becomes large. As another example, an angular velocity value, a direction of an angular velocity, an acceleration value of the angular velocity, an angle, displacement information, stress, and the like corresponding to the posture signal also change. When the user stands up from a squatting state to a standing state, the amplitude information corresponding to the myoelectric signal, and the angular velocity value, the direction of the angular velocity, the acceleration value of the angular velocity, the angle, the displacement information, and the stress corresponding to the posture signal also change. Based on this case, the processing module 220 may segment the motion signal of the user based on the feature information corresponding to the myoelectric signal or the feature information corresponding to the posture signal. For details about segmenting the motion signal based on the feature information corresponding to the myoelectric signal or the feature information corresponding to the posture signal, reference may be made to FIG. 7 and FIG. 8 in this disclosure and related descriptions thereof.

Step 620: Monitor the motion of the user based on at least one motion signal segment.

This step may be performed by the processing module 220 and/or the processing device 110. In some exemplary embodiments, monitoring the motion of the user based on the at least one motion signal segment may include: performing matching based on the at least one motion signal segment and at least one preset motion signal segment, and determining a motion type of the user. The at least one preset motion signal segment refers to preset standard motion signals corresponding to different motions in a database. In some exemplary embodiments, the motion type of the user may be determined by determining a degree of matching between the at least one motion signal segment and the at least one preset motion signal segment. Further, it may be determined whether a degree of matching between the motion signal and a preset motion signal is within a first matching threshold range (for example, greater than 80%), and if yes, the motion type of the user may be determined based on a motion type corresponding to the preset motion signal. In some exemplary embodiments, monitoring the motion during moving of the user based on the at least one motion signal segment may further include: performing matching based on feature information corresponding to at least one myoelectric signal segment and feature information corresponding to a myoelectric signal in the at least one preset motion signal segment, and determining the motion type of the user. For example, a degree of matching between one or more pieces of feature information (for example, frequency information and amplitude information) in one myoelectric signal segment and one or more pieces of feature information in one preset motion signal segment may be calculated separately, whether a weighted degree of matching or an average degree of matching of the one or more pieces of feature information is within the first matching threshold range is determined, and if yes, the motion type of the user may be determined based on the motion type corresponding to the preset motion signal. In some exemplary embodiments, monitoring the motion during moving of the user based on the at least one motion signal segment may further include: performing matching based on feature information corresponding to at least one posture signal segment and feature information corresponding to a posture signal in the at least one preset motion signal segment, and determining the motion type of the user. For example, a degree of matching between one or more pieces of feature information (for example, an angular velocity value, a direction of an angular velocity, an acceleration value of the angular velocity, an angle, displacement information, and stress) in one posture signal segment and one or more pieces of feature information in one preset motion signal segment may be calculated separately, whether a weighted degree of matching or an average degree of matching of the one or more pieces of feature information is within the first matching threshold range may be determined, and if yes, the motion type of the user is determined based on the motion type corresponding to the preset motion signal. In some exemplary embodiments, monitoring the motion during moving of the user based on the at least one motion signal segment may further include: performing matching based on feature information corresponding to a myoelectric signal and feature information corresponding to a posture signal in the at least one motion signal segment, and feature information corresponding to a myoelectric signal and feature information corresponding to a posture signal in the at least one preset motion signal segment, and determining the motion type of the user.

In some exemplary embodiments, the monitoring of the motion during moving of the user based on the at least one motion signal segment may include: performing matching based on the at least one motion signal segment and the at least one preset motion signal segment, and determining motion quality of the user. Further, if the degree of matching between the motion signal and the preset motion signal is within a second matching threshold range (for example, greater than 90%), the motion quality during moving of the user conforms to a standard. In some exemplary embodiments, the determining of the motion during moving of the user based on the at least one motion signal segment may include: performing matching based on one or more pieces of feature information in the at least one motion signal segment and one or more pieces of feature information in the at least one preset motion signal segment, and determining the motion quality during moving of the user. It should be noted that one motion signal segment may be a motion signal of a full motion or a motion signal of a partial motion in a full motion. In some exemplary embodiments, for a complex full motion, there are different force generation modes at different stages of the full motion, that is, there are different motion signals at different stages of the motion, and real-time monitoring of the motion of the user may be improved by monitoring the motion signals at different stages of the full motion.

FIG. 7 is an exemplary flowchart for motion signal segmentation according to some exemplary embodiments of this disclosure. As shown in FIG. 7, the process 700 may include the following steps.

Step 710: Determine, based on a time domain window of a myoelectric signal or a posture signal, at least one target feature in the time domain window based on a preset condition.

In some exemplary embodiments, this step may be performed by the processing module 220 and/or the processing device 110. The time domain window of the myoelectric signal contains the myoelectric signal within a time range, and the time domain window of the posture signal contains the posture signal within the same time range. The target feature refers to a signal with a target feature in a motion signal, and the target feature can represent a stage of a motion of a user. For example, when the user performs seated chest fly, two arms of the user are initially in a left-right extension state in a horizontal direction, then the arms start to internally rotate, then the arms are closed, and finally the arms return to the extension state again in the horizontal direction. This process is a full seated chest fly motion. When the user performs a seated chest fly motion, feature information corresponding to a myoelectric signal or a posture signal at each stage may be different, and a target feature corresponding to the stage of the motion of the user may be determined by analyzing the feature information (for example, amplitude information and frequency information) corresponding to the myoelectric signal or the feature information (for example, an angular velocity value, a direction of an angular velocity, an acceleration value of the angular velocity, an angle, displacement information, and stress) corresponding to the posture signal. In some exemplary embodiments, one or more target features may be determined in the time domain window based on the preset condition. In some exemplary embodiments, the preset condition may include one or more of the following: the direction of the angular velocity corresponding to the posture signal changes, the angular velocity corresponding to the posture signal is greater than or equal to an angular velocity threshold, the angle corresponding to the posture signal reaches an angle threshold, a variation of the angular velocity value corresponding to the posture signal is an extreme value, and the amplitude information corresponding to the myoelectric signal is greater than or equal to a myoelectric threshold. In some exemplary embodiments, target features of different stages of one motion may correspond to different preset conditions. For example, in seated chest fly, the preset condition for the target feature when the two arms of the user are in the left-right extension state in the horizontal direction and then the arms start to internally rotate, may be different from the preset condition for the target feature when the two arms are closed. In some exemplary embodiments, target features of different motions may correspond to different preset conditions. For example, the seated chest fly motion may be different from a biceps lifting motion, and preset conditions corresponding to preset target points of the two motions are also different. For content of examples of the preset conditions, reference may be made to descriptions of a motion start point, a motion middle point, and a motion end point in this disclosure.

In some exemplary embodiments, at least one target feature may be further determined in the time domain window based on the preset condition based on the time domain window of the myoelectric signal and the time domain window of the posture signal. Herein, the target feature may be determined with reference to the feature information (for example, the amplitude information) corresponding to the myoelectric signal and the feature information (for example, the angular velocity value, the direction of the angular velocity, the acceleration value of the angular velocity, and the angle) corresponding to the posture signal.

Step 720: Segment the motion signal based on the at least one target feature.

In some exemplary embodiments, step 720 may be performed by the processing module 220 and/or the processing device 110. In some exemplary embodiments, there may be one or more target features in the myoelectric signal or the posture signal, and the motion signal may be segmented based on the one or more target features. It should be noted that the target feature may correspond to one or more motion stages. When the target feature corresponds to a plurality of motion stages, the motion signal may be segmented by using a plurality of target features for reference. For example, the motion stage corresponding to the target feature may include a motion start point and a motion end point, where the motion start point is before the motion end point, and the motion signal between the motion start point and a next motion start point may be considered as a motion signal segment herein.

In some exemplary embodiments, the target feature may include one or more of a motion start point, a motion middle point, and a motion end point.

In some exemplary embodiments, at least one group of the motion start point, the motion middle point, and the motion end point in the motion signal may be determined repeatedly, and the motion signal is segmented based on the at least one group of the motion start point, the motion middle point, and the motion end point used as target features.

It should be noted that the foregoing segmentation and monitoring of the motion signal based on the motion start point, the motion middle point, and the motion end point used as a group of target features is merely illustrative. In some exemplary embodiments, the motion signal of the user may be further segmented and monitored based on any one or more of the motion start point, the motion middle point, and the motion end point used as target features. For example, the motion signal may be further segmented and monitored by using the motion start point as a target feature. As another example, the motion signal may be further segmented and monitored by using the motion start point and the motion end point as a group of target features, and other time points or time ranges that can function as target features shall all fall within the protection scope of this disclosure.

FIG. 8 is a schematic diagram of motion signal segmentation according to some exemplary embodiments of this disclosure. In FIG. 8, a horizontal coordinate may represent the user’s motion time, and a vertical coordinate may represent amplitude information of a myoelectric signal at a corresponding muscle part (for example, pectoralis major) when the user performs seated chest fly exercise. FIG. 8 further includes an angular velocity variation curve and an Euler angle variation curve corresponding to a posture signal at a wrist position during moving of the user, where the angular velocity variation curve may be used to represent a speed change during moving of the user, and the Euler angle curve is used to represent a position of a body part during moving of the user. As shown in FIG. 8, a point A1 is determined as a motion start point based on a preset condition. Specifically, a direction of an angular velocity at a time point after the user motion start point A1 is changed in comparison with a direction of an angular velocity at a time point before the motion start point A1. Further, an angular velocity value at the motion start point A1 is approximately 0, and an acceleration value of the angular velocity at the motion start point A1 is greater than 0.

With reference to FIG. 8, a point B1 is determined as a motion middle point based on the preset condition. Specifically, a direction of an angular velocity at a time point after the user motion middle point B1 is changed in comparison with a direction of an angular velocity at a time point before the motion middle point Bl, and an angular velocity value at the motion middle point B1 is approximately 0, where a direction of an angular velocity at the motion middle point B1 is opposite to the direction of the angular velocity at the motion start point Al. In addition, in the myoelectric signal (“myoelectric signal” shown in FIG. 8), an amplitude corresponding to the motion middle point B1 is greater than a myoelectric threshold.

With continued reference to FIG. 8, a point C1 is determined as a motion end point based on the preset condition. Specifically, a variation of an angular velocity value at the motion end point C1 is an extreme value from the motion start point A1 to the motion end point C1. In some exemplary embodiments, motion segmentation shown in FIG. 8 may be completed in the process 700, and the motion signal from the motion start point A1 to the motion end point C1 shown in FIG. 8 may be considered as a signal segment during moving of the user.

During obtaining of the motion signal of the user, other physiological parameter information (for example, a heart rate signal) of the user, and external conditions such as relative motion or pressure between the obtaining module 210 and the human body in the moving process may affect quality of the motion signal, for example, may cause an abruption in the myoelectric signal, thus affecting monitoring of the motion of the user. For ease of description, the abrupt myoelectric signal may be described by using a singularity. For example, the singularity may include a burr signal or a discontinuous signal. In some exemplary embodiments, the monitoring of the motion of the user based on the at least feature information corresponding to the myoelectric signal or the feature information corresponding to the posture signal may further include: preprocessing the myoelectric signal in a frequency domain or time domain, obtaining, based on the preprocessed myoelectric signal, the feature information corresponding to the myoelectric signal, and monitoring the motion of the user during moving of the user based on the feature information corresponding to the myoelectric signal or the feature information corresponding to the posture signal. In some exemplary embodiments, the preprocessing of the myoelectric signal in the frequency domain or time domain may include filtering the myoelectric signal in the frequency domain to select or retain components within a specific frequency range in the myoelectric signal in the frequency domain.

In some exemplary embodiments, the preprocessing of the myoelectric signal in the frequency domain or time domain may further include performing signal correction processing on the myoelectric signal in the time domain. The signal correction processing refers to correcting the singularity (for example, the burr signal or the discontinuous signal) in the myoelectric signal. In some exemplary embodiments, the performing of the signal correction processing on the myoelectric signal in the time domain may include determining the singularity in the myoelectric signal, that is, determining the abrupt signal in the myoelectric signal. The singularity may refer to that an amplitude of the myoelectric signal is abrupt within a certain time period, resulting in discontinuity of the signal. As another example, a pattern of the myoelectric signal is smooth, the amplitude of the myoelectric signal is not abrupt, but a first-order differential of the myoelectric signal is abrupt, and the first-order differential is discontinuous. In some exemplary embodiments, a method for determining the singularity in the myoelectric signal may include but is not limited to one or more of a Fourier transform, a wavelet transform, a fractal dimension, and the like. In some exemplary embodiments, the performing of the signal correction processing on the myoelectric signal in time domain may include removing the singularity in the myoelectric signal, for example, deleting the singularity and signals within a time range around the singularity. Alternatively, the performing of the signal correction processing on the myoelectric signal in time domain may include modifying the singularity in the myoelectric signal based on feature information of the myoelectric signal within a specific time range, for example, adjusting an amplitude of the singularity based on signals around the singularity. In some exemplary embodiments, the feature information of the myoelectric signal may include one or more of amplitude information and statistical information of the amplitude information. The statistical information of the amplitude information (also referred to as amplitude entropy) may refer to a distribution of the amplitude information of the myoelectric signal in time domain. In some exemplary embodiments, after a position (for example, a corresponding time point) of the singularity in the myoelectric signal is determined by using a signal processing algorithm (for example, the Fourier transform, the wavelet transform, or the fractal dimension), the singularity may be modified based on a myoelectric signal within a particular time range before or after the position of the singularity. For example, when the singularity is an abrupt valley, the myoelectric signal at the abrupt valley may be supplemented based on feature information (for example, amplitude information and statistical information of the amplitude information) of the myoelectric signal within a specific time range (for example, 5 ms to 60 ms) before or after the abrupt valley.

Exemplarily illustrated by using a singularity as a burr signal, FIG. 9 is an exemplary flowchart for preprocessing a myoelectric signal according to some exemplary embodiments of this disclosure. As shown in FIG. 9, the process 900 may include the following steps.

Step 910: Select, based on a time domain window of the myoelectric signal, different time windows from the time domain window of the myoelectric signal, where the different time windows cover different time ranges respectively.

In some exemplary embodiments, this step may be performed by the processing module 220 and/or the processing device 110. In some exemplary embodiments, the different windows may include at least one specific window. The specific window may be a window selected from the time domain window and having a specific duration. For example, when a duration of the time domain window of the myoelectric signal is 3 s, the duration of the specific window may be 100 ms. In some exemplary embodiments, the specific window may include a plurality of different time windows. For illustration only, the specific window may include a first time window and a second time window. The first time window may be a window of a corresponding partial duration in the specific window. For example, when the duration of the specific window is 100 ms, the duration of the first time window may be 80 ms. The second time window may be another window of a corresponding partial duration in the specific window. For example, when the specific window is 100 ms, the second time window may be 20 ms. In some exemplary embodiments, the first time window and the second time window may be continuous time windows in the same specific window. In some exemplary embodiments, the first time window and the second time window may alternatively be two discrete or overlapping time windows in the same specific window. In some exemplary embodiments, the processing module 220 may slide and update the specific window sequentially by a specific duration from a start time of the time domain window of the myoelectric signal based on the time domain window of the myoelectric signal, and may continue to divide the updated specific window into the first time window and the second time window. The specific duration herein may be less than 1 s, 2 s, 3 s, or the like. For example, the processing module 220 may select a specific window having a specific duration of 100 ms, and divide the specific window into the first time window of 80 ms and the second time window of 20 ms. Further, the specific window may be slid and updated along a time direction. A sliding distance herein may be the duration of the second time window (for example, 20 ms), or may be another suitable duration, for example, 30 ms or 40 ms.

Step 920: Determine a burr signal based on feature information corresponding to the myoelectric signal in different time windows.

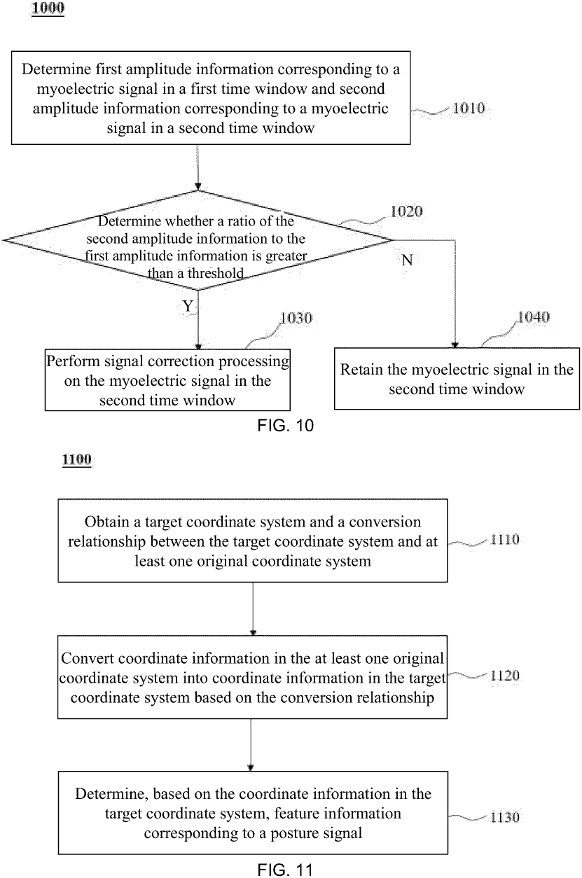

In some exemplary embodiments, this step may be performed by the processing module 220 and/or the processing device 110. In some exemplary embodiments, the feature information corresponding to the myoelectric signal may include at least one of amplitude information and statistical information of the amplitude information. In some exemplary embodiments, the processing module 220 may obtain the amplitude information or the statistical information of the amplitude information corresponding to the myoelectric signal in different time windows (for example, the first time window and the second time window), and determine a position of the burr signal. For detailed descriptions about determining the position of the burr signal based on the feature information corresponding to the myoelectric signal in different time windows, reference may be made to FIG. 10 and the related descriptions thereof.

FIG. 10 is an exemplary flowchart for removing a burr signal according to some exemplary embodiments of this disclosure. As shown in FIG. 10, the process 1000 may include the following steps.

Step 1010: Determine first amplitude information corresponding to a myoelectric signal in a first time window and second amplitude information corresponding to a myoelectric signal in a second time window.

In some exemplary embodiments, this step may be performed by the processing module 220 and/or the processing device 110. In some exemplary embodiments, the processing module 220 may select a duration of the first time window and a duration of the second time window, and extract the first amplitude information corresponding to the myoelectric signal within the duration of the first time window and the second amplitude information corresponding to the myoelectric signal within the duration of the second time window. In some exemplary embodiments, the first amplitude information may include an average amplitude of the myoelectric signal in the first time window and the second amplitude information may include an average amplitude of the myoelectric signal in the second time window. For example, the processing module 220 may select an 80 ms duration of the first time window and extract the first amplitude information corresponding to the myoelectric signal in the first time window, and the processing module 220 may select a 20 ms duration of the second time window and extract the second amplitude information corresponding to the myoelectric signal in the second time window.

In some exemplary embodiments, the duration of the first time window and the duration of the second time window are selected in relation to a shortest burr signal duration and a calculation amount of a system. In some exemplary embodiments, the duration of the first time window and the duration of the second time window may be selected based on a feature of a burr signal. A duration of an electrocardio burr signal is 40 ms to 100 ms, a time interval between two burr signals in an electrocardio signal may be approximately 1 s, two sides of peak points of the burr signal are basically symmetrical, an amplitude distribution on two sides of the burr signal is relatively even, and so on. In some exemplary embodiments, when the burr signal is an electrocardio signal, a duration shorter than the duration of the burr signal, for example, one half of the duration of the burr signal, may be selected as the duration of the second time window, and the duration of the first time window may be longer than the duration of the second time window, for example, four times the duration of the second time window. In some exemplary embodiments, the duration of the first time window only needs to be within a range of a burr signal interval (approximately 1 s) minus the duration of the second time window. It should also be noted that the foregoing selection of the duration of the first time window and the duration of the second time window is not limited to the foregoing description, as long as a sum of the duration of the second time window and the duration of the first time window is less than a time interval between two adjacent burr signals, or the duration of the second time window is shorter than a duration of a single burr signal, or an amplitude of the myoelectric signal in the second time window and an amplitude of the myoelectric signal in the first time window can be well discriminated.

Step 1020: Determine whether a ratio of the second amplitude information to the first amplitude information is greater than a threshold.

In some exemplary embodiments, this step may be performed by the processing module 220 and/or the processing device 110. In some exemplary embodiments, the processing module 220 may determine whether the ratio of the second amplitude information corresponding to the myoelectric signal in the second time window to the first amplitude information corresponding to the myoelectric signal in the first time window is greater than the threshold. The threshold herein may be stored in a memory or a hard disk of the wearable device 130, or may be stored in the processing device 110, or may be adjusted based on an actual situation. In some exemplary embodiments, if the processing module 220 determines that the ratio of the second amplitude information to the first amplitude information is greater than the threshold, step 1030 may be performed after step 1020. In some exemplary embodiments, if the processing module 220 determines that the ratio of the second amplitude information to the first amplitude information is not greater than the threshold, step 1040 may be performed after step 1020.

Step 1030: Perform signal correction processing on the myoelectric signal in the second time window.

In some exemplary embodiments, this step may be performed by the processing module 220 and/or the processing device 110. In some exemplary embodiments, the processing module 220 may perform signal correction processing on the myoelectric signal in the second time window based on a result of determining a relationship between the ratio of the second amplitude information to the first amplitude information and the threshold in step 1020. For example, in some exemplary embodiments, if the ratio of the second amplitude information to the first amplitude information is greater than the threshold, the myoelectric signal in the second time window corresponding to the second amplitude information is a burr signal. In some exemplary embodiments, processing the myoelectric signal in the second time window may include performing signal correction processing on the myoelectric signal in the second time window based on a myoelectric signal within a specific time range before or after the second time window.

Step 1040: Retain the myoelectric signal in the second time window.

In some exemplary embodiments, this step may be performed by the processing module 220 and/or the processing device 110. In some exemplary embodiments, the processing module 220 may retain the myoelectric signal in the second time window based on the result of determining the relationship between the ratio of the second amplitude information to the first amplitude information and the threshold in step 1020. For example, in some exemplary embodiments, if the ratio of the second amplitude information to the first amplitude information is not greater than the threshold, the myoelectric signal in the second time window corresponding to the second amplitude information is a normal myoelectric signal, and the normal myoelectric signal may be retained, that is, the myoelectric signal in the second time window is retained.

It should be noted that, because charge is gradually accumulated during force generation by a muscle of the user, and the amplitude of the myoelectric signal gradually increases, in absence of a burr signal, amplitudes of myoelectric signals in two adjacent time windows (for example, the first time window and the second time window) do not change abruptly. In some exemplary embodiments, the determining and removing of the burr signal in the myoelectric signal based on the process 1000 may enable real-time processing of the burr signal, thereby enabling the wearable device 130 or the mobile terminal device 140 to feedback a moving status of the user to the user in real time to assist the user in performing more scientific motion.

In some exemplary embodiments, the duration corresponding to the first time window may be longer than the duration corresponding to the second time window. In some exemplary embodiments, a specific duration corresponding to a specific window may be shorter than 1 s. In some exemplary embodiments, a ratio of the duration corresponding to the first time window to the duration corresponding to the second time window may be greater than 2. In some exemplary embodiments, the selection of the duration corresponding to the first time window, the duration corresponding to the second time window, and the specific duration corresponding to the specific window, on one hand, can ensure that a shortest burr signal duration (for example, 40 ms) can be removed and that a signal-to-noise ratio is high, and on the other hand, can make the calculation amount of the system relatively small, reduce repeated calculation of the system, and reduce time complexity, so that calculation efficiency and calculation accuracy of the system may be improved.

In some exemplary embodiments, a posture signal may be obtained by a posture sensor in the wearable device 130. Posture sensors in the wearable device 130 may be distributed on four limbs of the human body (for example, arms and legs), the torso of the human body (for example, the chest, abdomen, back, and waist), the head of the human body, and the like. The posture sensors can capture posture signals at other parts of the human body, such as the limbs and the torso. In some exemplary embodiments, a posture sensor may also be a sensor having an attitude and heading reference system (AHRS) with a posture fusion algorithm. The posture fusion algorithm may fuse data of a nine-axis inertial measurement unit (IMU) having a three-axis acceleration sensor, three-axis angular velocity sensor, and a three-axis geomagnetic sensor into an Euler angle or quaternion, to obtain a posture signal of a body part of the user in which the posture sensor is located. In some exemplary embodiments, the processing module 220 and/or the processing device 110 may determine, based on the posture signal, the feature information corresponding to the posture. In some exemplary embodiments, the feature information corresponding to the posture signal may include but is not limited to an angular velocity value, a direction of an angular velocity, an acceleration value of the angular velocity, and the like. In some exemplary embodiments, the posture sensor may be a strain sensor, and the strain sensor may obtain a bending direction and a bending angle at the user’s joint to obtain a posture signal during moving of the user. For example, the strain sensor may be disposed at a knee joint of the user, during moving of the user, a body part of the user acts on the strain sensor, and a bending direction and a bending angle at the knee joint of the user may be calculated based on a resistance or length change of the strain sensor, to obtain a posture signal of a leg of the user. In some exemplary embodiments, the posture sensor may further include a fiber optic sensor, and the posture signal may be represented by a change of a direction of light after bending at the fiber optic sensor. In some exemplary embodiments, the posture sensor may alternatively be a magnetic flux sensor, and the posture signal may be represented by a change of a magnetic flux. It should be noted that the type of the posture sensor is not limited to the foregoing sensors, but may also be other sensors. All sensors capable of obtaining a posture signal of a user shall fall within the scope of the posture sensor in this disclosure.

FIG. 11 is an exemplary flowchart for determining feature information corresponding to a posture signal according to some exemplary embodiments of this disclosure. As shown in FIG. 11, the process 1100 may include the following steps.

Step 1110: Obtain a target coordinate system and a conversion relationship between the target coordinate system and at least one original coordinate system.

In some exemplary embodiments, this step may be performed by the processing module 220 and/or the processing device 110. In some exemplary embodiments, the original coordinate system may be a coordinate system corresponding to a posture sensor disposed on a human body. When a user uses the wearable device 130, various posture sensors in the wearable device 130 are distributed at different parts of the human body, so that installation angles of the various posture sensors on the human body may be different, and the posture sensors at different parts respectively use their coordinate systems as original coordinate systems. Therefore, the posture sensors at different parts may have different original coordinate systems. In some exemplary embodiments, a posture signal obtained by each posture sensor may be an expression in an original coordinate system corresponding to the posture sensor. Posture signals in different original coordinate systems are converted into posture signals in a same coordinate system (that is, the target coordinate system), so that relative motion between different parts of the human body is determined. In some exemplary embodiments, the target coordinate system is a human body coordinate system established based on the human body. For example, in the target coordinate system, a length direction of a torso of the human (that is, a direction perpendicular to a transverse plane of the human body) may be used as a Z-axis, a front-back direction of the torso of the human (that is, a direction perpendicular to a coronal plane of the human body) may be used as an X-axis, and a left-right direction of the torso of the human body (that is, a direction perpendicular to a sagittal plane of the human body) may be used as a Y-axis. In some exemplary embodiments, a conversion relationship exists between the target coordinate system and the original coordinate system, and by using the conversion relationship, coordinate information in the original coordinate system can be converted into coordinate information in the target coordinate system. In some exemplary embodiments, the conversion relationship may be represented by one or more rotation matrices. For details about determining the conversion relationship between the target coordinate system and the original coordinate system, reference may be made to FIG. 13 in this disclosure and related descriptions thereof.

Step 1120: Convert coordinate information in the at least one original coordinate system into coordinate information in the target coordinate system based on the conversion relationship.

In some exemplary embodiments, this step may be performed by the processing module 220 and/or the processing device 110. The coordinate information in the original coordinate system is three-dimensional coordinate information in the original coordinate system. The coordinate information in the target coordinate system is three-dimensional coordinate information in the target coordinate system. For illustration only, for coordinate information v₁ in the original coordinate system, the coordinate information in the original coordinate system may be converted into coordinate information ν₂ in the target coordinate system based on the conversion relationship. Specifically, the conversion between the coordinate information ν₁ and the coordinate information ν₂ may be performed by using a rotation matrix. The rotation matrix herein may be understood as the conversion relationship between the original coordinate system and the target coordinate system. Specifically, the coordinate information ν₁ in the original coordinate system may be converted into the coordinate information ν₁-1 by using a first rotation matrix, the coordinate information ν₁-1 may be converted into the coordinate information ν₁-2 by using a second rotation matrix, the coordinate information ν₁-2 may be converted into the coordinate information ν₁-3 by using a third rotation matrix, and the coordinate information ν₁-3 is the coordinate information ν₂ in the target coordinate system. It should be noted that the rotation matrix is not limited to the first rotation matrix, the second rotation matrix, and the third rotation matrix described above, and may further include fewer or more rotation matrices. In some exemplary embodiments, the rotation matrix may alternatively be one rotation matrix or a combination of a plurality of rotation matrices.

Step 1130: Determine, based on the coordinate information in the target coordinate system, feature information corresponding to a posture signal.

In some exemplary embodiments, this step may be performed by the processing module 220 and/or the processing device 110. In some exemplary embodiments, the determining, based on the coordinate information in the target coordinate system, of the feature information corresponding to the posture signal of the user may include: determining, based on a plurality of pieces of coordinate information in the target coordinate system during moving of the user, the feature information corresponding to the posture signal of the user. For example, when the user performs seated chest fly exercise, forward lifting of arms by the user may correspond to first coordinate information in the target coordinate system, opening of the arms to align with the torso on the same plane by the user may correspond to second coordinate information in the target coordinate system, and the feature information corresponding to the posture signal of the user may be calculated based on the first coordinate information and the second coordinate information, for example, an angular velocity, a direction of the angular velocity, and an acceleration value of the angular velocity.

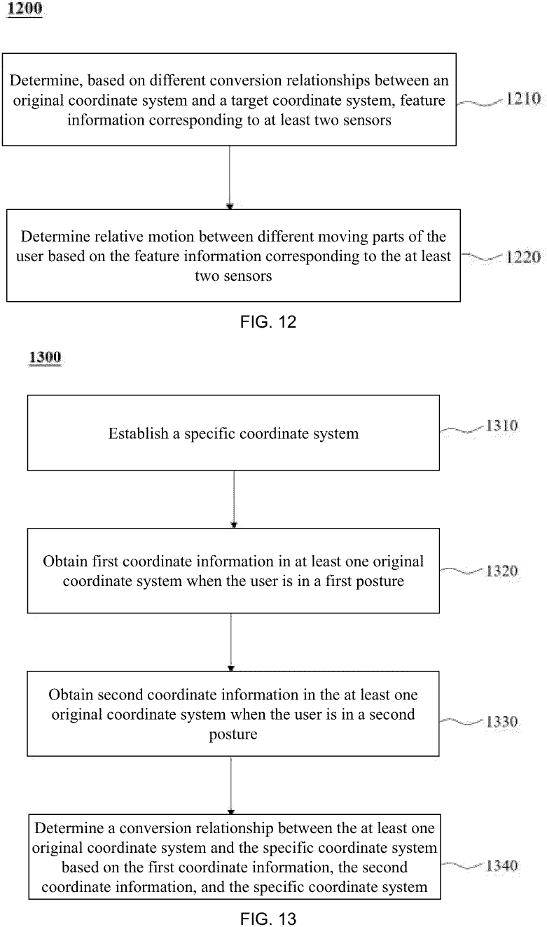

In some exemplary embodiments, the relative motion between different parts of the body of the user may be further determined by using feature information corresponding to posture sensors located at different positions of the body of the user. For example, the relative motion between the arms and the torso during moving of the user may be determined by using feature information corresponding to posture sensors at the arms of the user and feature information corresponding to a posture sensor at the torso of the user. FIG. 12 is an exemplary flowchart for determining relative motion between different moving parts of a user according to some exemplary embodiments of this disclosure. As shown in FIG. 12, the process 1200 may include the following steps.

Step 1210: Determine, based on different conversion relationships between an original coordinate system and a target coordinate system, feature information corresponding to at least two sensors.

In some exemplary embodiments, this step may be performed by the processing module 220 and/or the processing device 110. In some exemplary embodiments, because installation positions of different sensors on the human body are different, there may be different conversion relationships between original coordinate systems corresponding to the original coordinate systems and the target coordinate system. In some exemplary embodiments, the processing device 110 may convert coordinate information in original coordinate systems corresponding to sensors at different parts of a user (for example, a forearm, an upper arm, and a torso) into coordinate information in the target coordinate system, so that feature information corresponding to at least two sensors can be obtained. Descriptions about converting coordinate information in an original coordinate system into coordinate information in the target coordinate system may be found elsewhere in this disclosure, for example, in FIG. 11. Details will not described herein again.

Step 1220: Determine relative motion between different moving parts of the user based on the feature information corresponding to the at least two sensors.

In some exemplary embodiments, this step may be performed by the processing module 220 and/or the processing device 110. In some exemplary embodiments, the moving parts may be limbs that may move independently, for example, forearms, upper arms, calves, and thighs. For illustration only, when the user performs dumbbell arm lifting exercise, a combination of coordinate information in the target coordinate system corresponding to a sensor disposed at a forearm part and coordinate information in the target coordinate system corresponding to a sensor disposed at an upper arm part may be used to determine relative motion between the forearm and the upper arm of the user, so that a dumbbell arm lifting motion of the user can be determined.

In some exemplary embodiments, a plurality of sensors of a same type or different types may also be disposed at a same moving part of the user, and coordinate information in the original coordinate systems corresponding to the plurality of sensors of the same type or different types may be respectively converted into coordinate information in the target coordinate system. For example, a plurality of sensors of a same type or different types may be disposed at different positions of a forearm part of the user, and a plurality of pieces of coordinate information in the target coordinate system corresponding to the plurality of sensors of the same type or different types may simultaneously represent a moving motion of the forearm of the user. For example, the coordinate information in the target coordinate system corresponding to the plurality of sensors of the same type may be averaged to improve accuracy of the coordinate information of the moving part during moving of the user. As another example, a fusion algorithm (for example, Kalman filtering) may be applied to the coordinate information in the coordinate systems corresponding to the plurality of sensors of different types, to obtain the coordinate information in the target coordinate system.

FIG. 13 is an exemplary flowchart for determining a conversion relationship between an original coordinate system and a specific coordinate system according to some exemplary embodiments of this disclosure. In some exemplary embodiments, a process of determining a conversion relationship between an original coordinate system and a specific coordinate system may also be referred to as a calibration process. As shown in FIG. 13, the process 1300 may include the following steps.

Step 1310: Establish a specific coordinate system.

In some exemplary embodiments, this step may be performed by the processing module 220 and/or the processing device 110. In some exemplary embodiments, a conversion relationship between at least one original coordinate system and a target coordinate system may be obtained through a calibration process. The specific coordinate system is a reference coordinate system used to determine the conversion relationship between the original coordinate system and the target coordinate system in the calibration process. In some exemplary embodiments, the established specific coordinate system may use a length direction of a torso of a standing human body as a Z-axis, use a front-back direction of the human body as an X-axis, and use a left-right direction of the torso of the human body as a Y-axis. In some exemplary embodiments, the specific coordinate system is related to an orientation of the user in the calibration process. For example, in the calibration process, when the body of the user faces a fixed direction (for example, north), a direction (north) in front of the human body is the X-axis, and the direction of the X-axis in the calibration process is fixed.

Step 1320: Obtain first coordinate information in at least one original coordinate system when the user is in a first posture.

In some exemplary embodiments, this step may be performed by the obtaining module 210. The first posture may be a posture in which the user keeps approximately standing. The obtaining module 210 (for example, a sensor) may obtain the first coordinate information in the original coordinate system based on the first posture of the user.

Step 1330: Obtain second coordinate information in the at least one original coordinate system when the user is in a second posture.

In some exemplary embodiments, this step may be performed by the obtaining module 210. The second posture may be a posture in which a body part (for example, an arm) of the user at which the sensor is located is tilted forward. In some exemplary embodiments, the obtaining module 210 (for example, a sensor) may obtain the second coordinate information in the original coordinate system based on the second posture of the user (for example, a forward tilt posture).

Step 1340: Determine a conversion relationship between the at least one original coordinate system and the specific coordinate system based on the first coordinate information, the second coordinate information, and the specific coordinate system.

In some exemplary embodiments, this step may be performed by the processing module 220 and/or the processing device 110. In some exemplary embodiments, a first rotation matrix may be determined by using the first coordinate information corresponding to the first posture. In the first posture, because Euler angles in X and Y directions of the specific coordinate system in a ZYX rotation sequence are 0, and Euler angles in X and Y directions of the original coordinate system are not necessarily 0, the first rotation matrix is a rotation matrix obtained by inversely rotating the original coordinate system around the X-axis and then inversely rotating around the Y-axis. In some exemplary embodiments, a second rotation matrix may be determined by using the second coordinate information of the second posture (for example, forward tilt of a body part at which the sensor is located). Specifically, in the second posture, because Euler angles in Y and Z3 directions of the specific coordinate system in the ZYX rotation sequence are 0, and Euler angles in Y and Z3 directions of the original coordinate system are not necessarily 0, the second rotation matrix is a rotation matrix obtained by inversely rotating the original coordinate system around the Y direction and then inversely rotating around the Z3 direction. The conversion relationship between the original coordinate system and the specific coordinate system may be determined by using the foregoing first rotation matrix and second rotation matrix. In some exemplary embodiments, when there are a plurality of original coordinate systems (sensors), the method described above may be used to determine the conversion relationship between each original coordinate system and the specific coordinate system.

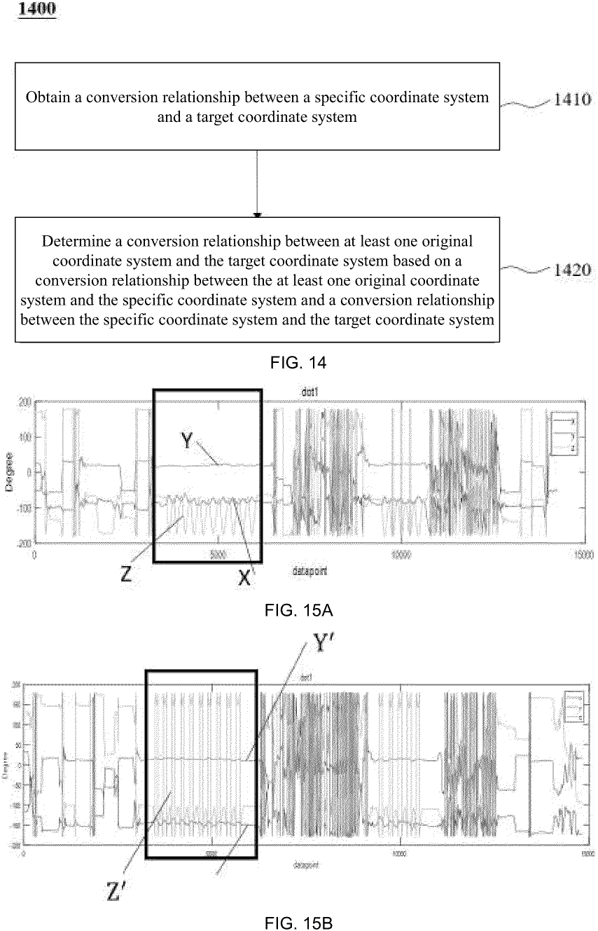

FIG. 14 is an exemplary flowchart for determining a conversion relationship between an original coordinate system and a target coordinate system according to some exemplary embodiments of this disclosure. As shown in FIG. 14, the process 1400 may include the following steps.

Step 1410: Obtain a conversion relationship between a specific coordinate system and a target coordinate system.

In some exemplary embodiments, this step may be performed by the processing module 220 and/or the processing device 110. Because both the specific coordinate system and the target coordinate system use a length direction of a torso of a human body as a Z-axis, the conversion relationship between the specific coordinate system and the target coordinate system may be obtained by using a conversion relationship between the X-axis of the specific coordinate system and the X-axis of the target coordinate system and a conversion relationship between a Y-axis of the specific coordinate system and a Y-axis of the target coordinate system. For a principle of obtaining the conversion relationship between the specific coordinate relationship and the target coordinate system, reference may be made to FIG. 13 and related content thereof.

In some exemplary embodiments, the specific coordinate system may use the length direction of the torso of the human body as the Z-axis, and use a front-back direction of the human body as the X-axis. Because the front-back direction of the body of the user changes during moving of the user (for example, swivel motion) and may not be maintained in the calibrated coordinate system, it is necessary to determine a coordinate system that may rotate with the human body, that is, the target coordinate system. In some exemplary embodiments, the target coordinate system may change as an orientation of the user changes, but the X-axis of the target coordinate system may be always directly in front of the torso of the human body.

Step 1420: Determine a conversion relationship between at least one original coordinate system and the target coordinate system based on a conversion relationship between the at least one original coordinate system and the specific coordinate system and a conversion relationship between the specific coordinate system and the target coordinate system.

In some exemplary embodiments, this step may be performed by the processing module 220 and/or the processing device 110. In some exemplary embodiments, the processing device 110 may determine the conversion relationship between the at least one original coordinate system and the target coordinate system based on the conversion relationship between the at least one original coordinate system and the specific coordinate system that is determined in the process 1300 and the conversion relationship between the specific coordinate system and the target coordinate system that is determined in step 1410, so that coordinate information in the original coordinate system can be converted into coordinate information in the target coordinate system.

In some exemplary embodiments, a position of a posture sensor disposed on the wearable device 130 may vary and/or a posture sensor may be installed at a different angle on the human body. In this case, for the same motion performed by the user, posture data returned by the posture sensor may differ significantly.

FIG. 15A is an exemplary vector coordinate diagram of Euler angle data in an original coordinate system at a position of a forearm of a human body according to some exemplary embodiments of this disclosure. A line box may represent Euler angle data (coordinate information) in a corresponding original coordinate system at a position of a forearm when a user performs a same motion. As shown in FIG. 15A, Euler angle vector results in a Z-axis direction (shown as “Z” in FIG. 15A) within the line box approximately range from -180° to -80°, Euler angle vector results in a Y-axis direction (shown as “Y” in FIG. 15A) approximately fluctuate around 0°, and Euler angle vector results in an X-axis direction (shown as “X” in FIG. 15A) approximately fluctuate around -80°. A fluctuation range herein may be 20°.

FIG. 15B is an exemplary vector coordinate diagram of Euler angle data in an original coordinate system at another position of a forearm of a human body according to some exemplary embodiments of this disclosure. A line box may represent Euler angle data in a corresponding original coordinate system at another position of a forearm when a user performs a same motion (the same motion as that shown in FIG. 15A). As shown in FIG. 15B, Euler angle vector results in a Z-axis direction (shown as “Z′” in FIG. 15B) within the line box approximately range from -180° to 180°, Euler angle vector results in a Y-axis direction (shown as “Y′” in FIG. 15B) approximately fluctuate around 0°, and Euler angle vector results in an X-axis direction (shown as “X′” in FIG. 15B) approximately fluctuate around -150°. A fluctuation range herein may be 20°.

Euler angle data shown in FIG. 15A and FIG. 15B is Euler angle data (coordinate information) respectively obtained in the original coordinate system based on different positions of the forearm of the human body (it may also be understood that installation angles of posture sensors at the positions of the forearm of the human body may be different) when the user performs the same motion. By comparing FIG. 15A and FIG. 15B, it can be seen that in the case where the posture sensors are installed at different angles on the human body, when the user performs the same motion, Euler angle data returned by the posture sensors may differ greatly in the original coordinate system. For example, the Euler angle vector results in the Z-axis direction in FIG. 15A are approximately within a range of -180° to -80°), and the Euler angle vector results in the Z-axis direction in FIG. 15B are approximately within a range of -180° to 180°, and a great difference exists therebetween.

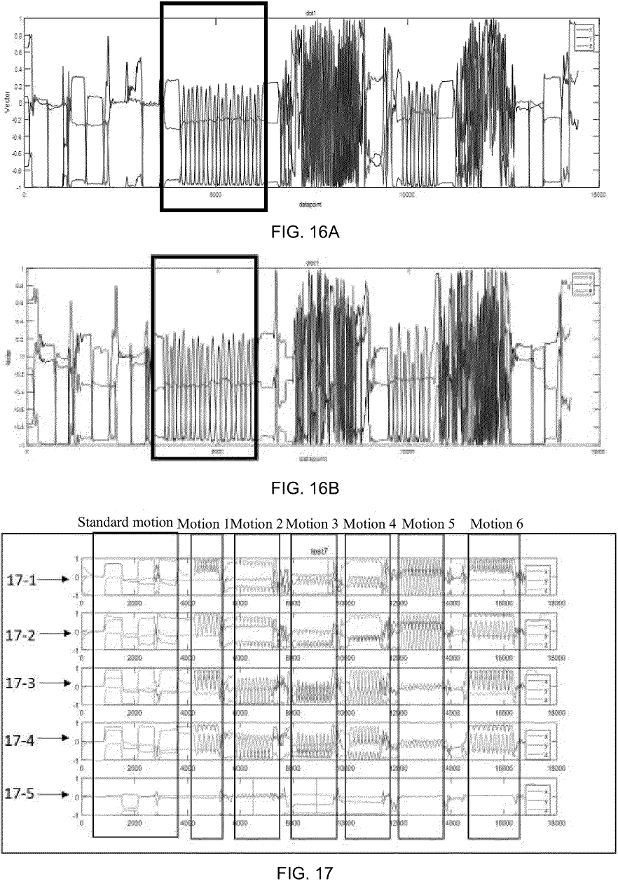

In some exemplary embodiments, Euler angle data in the original coordinate system corresponding to sensors at different installation angles may be converted into Euler angle data in a target coordinate system to facilitate analysis of posture signals for the sensors at different positions. For illustration only, a straight line on which a left arm is located may be abstracted as a unit vector pointing from an elbow to a wrist, and the unit vector is coordinate values in the target coordinate system. The target coordinate system herein is defined as follows: An axis pointing to a rear side of the human body is an X-axis, an axis pointing to a right side of the human body is a Y-axis, and an axis pointing above the human body is a Z-axis, which complies with a right-hand coordinate system. For example, coordinate values [-1, 0, 0] in the target coordinate system indicate that the arm is lifted forward; and coordinate values [0, -1, 0] in the target coordinate system indicate that the arm is lifted to the left. FIG. 16A is an exemplary vector coordinate diagram of Euler angle data in a target coordinate system at a position of a forearm of a human body according to some exemplary embodiments of this disclosure. FIG. 16A is a graph obtained based on the Euler angle data of the forearm in FIG. 15A after the Euler angle data of the original coordinates is converted into vector coordinates in the target coordinate system, where a line box may represent the Euler angle data in the target coordinate system at the position of the forearm when the user performs a motion. As shown in FIG. 16A, a forearm vector [x, y, z] within the line box reciprocates between a first position and a second position, where the first position is [0.2, -0.9, -0.38], and the second position is [0.1, -0.95, -0.3]. It should be noted that the first position and the second position may deviate by a small amount when the forearm reciprocates each time.

FIG. 16B is an exemplary vector coordinate diagram of Euler angle data in a target coordinate system at another position of a forearm of a human body according to some exemplary embodiments of this disclosure. FIG. 16B is a graph obtained based on the Euler angle data of the forearm in FIG. 15B after the Euler angle data of the original coordinates is converted into vector coordinates in the target coordinate system, where a line box may represent the Euler angle data in the target coordinate system at another position of the forearm when the user performs the same motion (the same motion as that shown in FIG. 16A). As shown in FIG. 16B, a forearm vector [x, y, z] also reciprocates between a first position and a second position, where the first position is [0.2, -0.9, -0.38], and the second position is [0.1, -0.95, -0.3].

With reference to FIG. 15A to FIG. 16B, it can be seen from FIG. 15A and FIG. 15B that because the installation positions of the two posture sensors may be different, Euler angles in the original coordinate systems may have a significant difference in a value range and a fluctuation form. After the coordinate information in the original coordinate systems corresponding to the two posture sensors is converted into corresponding vector coordinates in the target coordinate system (for example, the vector coordinates in FIG. 16A and FIG. 16B) respectively, two approximately same vector coordinates can be obtained. In other words, this method can make feature information corresponding to the posture signals not affected by the installation positions of the sensors. Specifically, it can be seen in FIG. 16A and FIG. 16B that the installation positions of the two posture sensors on the forearm are different, and after the foregoing coordinate conversion, the same vector coordinates are obtained, which can represent a process of reciprocal arm switching between two states of a state 1 (the arm lifting to the right) and a state 2 (the arm lifting to the front) during the seated chest fly.

FIG. 17 is a vector coordinate diagram of a limb vector in a target coordinate system according to some exemplary embodiments of this disclosure. As shown in FIG. 17, vector coordinates of posture sensors in the target coordinate system at positions of a left forearm (17-1), a right forearm (17-2), a left upper arm (17-3), a right upper arm (17-4), and a torso (17-5) of a human body may be represented from top to bottom respectively. Vector coordinates of various positions (for example, 17-1, 17-2, 17-3, 17-4, and 17-5) in the target coordinate system during moving of the human body are shown in FIG. 17. First 4200 points in FIG. 17 are calibration motions required to calibrate a limb, such as standing, torso forward, arm forward, and arm side-lifting. By using the calibration motions corresponding to the first 4200 points for calibration, original data captured by the posture sensor may be converted into Euler angle data in the target coordinate system. To facilitate data analysis, the data may be further converted into a coordinate vector of an arm vector in the target coordinate system. The target coordinate system herein has an X-axis pointing to the front of the torso, a Y-axis pointing to the left side of the torso, and a Z-axis pointing above the torso. The motions 1, 2, 3, 4, 5, and 6 among reciprocal motions from left to right in FIG. 17 are seated chest fly, pull down, seated chest press, seated shoulder press, barbell biceps curl, and seated chest fly respectively. It can be seen from FIG. 17 that different motions have different motion modes and can be clearly recognized by using a limb vector. In addition, the same motion also has a good repeatability. For example, the motion 1 and the motion 6 both represent a seated chest fly motion, and curves of the two motions have a good repeatability.

In some exemplary embodiments, the posture data (for example, Euler angles and angular velocities) directly output by the modules in the original coordinate system may be converted into posture data in the target coordinate system through the processes 1300 and 1400, so that highly consistent posture data (for example, Euler angles, angular velocities, and limb vector coordinates) may be obtained.

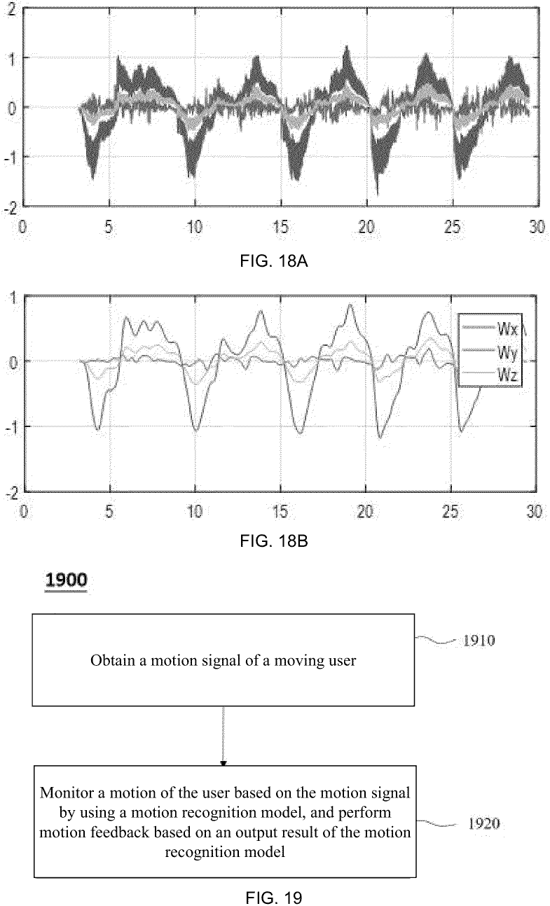

FIG. 18A is an exemplary vector coordinate diagram of an original angular velocity according to some exemplary embodiments of this disclosure. The original angular velocity may be understood in the case of converting the Euler angle data in the original coordinate system corresponding to the sensors at different installation angles into the Euler angle data in the target coordinate system. In some exemplary embodiments, factors such as shaking during moving of the user may affect a result of the angular velocity in the posture data. As shown in FIG. 18A, a vector coordinate curve of the original angular velocity is an obviously unsmooth curve under adverse impact of shaking or the like. For example, an abrupt signal exists in the vector coordinate curve of the original angular velocity, making the vector coordinate curve of the original angular velocity unsmooth. In some exemplary embodiments, for the adverse impact of shaking or the like on the result of the angular velocity, the angular velocity of shaking needs to be modified to obtain a smooth vector coordinate curve. In some exemplary embodiments, filtering processing may be performed on the original angular velocity by using a 1 Hz to 3 Hz low-pass filtering method. FIG. 18B is an exemplary result diagram of an angular velocity after filtering processing according to some exemplary embodiments of this disclosure. As shown in FIG. 18B, after 1 Hz to 3 Hz low-pass filtering processing is performed on the original angular velocity, the adverse impact of shaking or the like on the angular velocity (for example, an abrupt signal) can be eliminated, so that the vector coordinate diagram corresponding to the angular velocity can present a smooth curve. In some exemplary embodiments, 1 Hz to 3 Hz low-pass filtering processing of the angular velocity can effectively avoid the adverse impact of shaking or the like on the posture data (for example, Euler angles and angular velocities), thereby further facilitating a subsequent signal segmentation process. In some exemplary embodiments, the filtering processing may further filter out a power frequency signal and its harmonic signal, burr signal, and the like from a motion signal. It should be noted that the 1 Hz to 3 Hz low-pass filtering processing may cause a system delay, making a motion point of an obtained posture signal and a motion point of a real myoelectric signal staggered in time. Therefore, a system delay generated during the low-pass filtering processing is subtracted on a basis of the vector coordinate curve after the low-pass filtering processing, to ensure time synchronization of the posture signal and the myoelectric signal. In some exemplary embodiments, the system delay is associated with a center frequency of a filter, and when the posture signal and the myoelectric signal are processed by using different filters, the system delay is adaptively adjusted based on the center frequency of the filter. In some exemplary embodiments, because an angle range of an Euler angle is [-180°, +180°], when an actual Euler angle is not within this angle range, the obtained Euler angle may jump from -180° to +180° or from +180° to -180°. For example, when the angle is -181°, the Euler angle jumps to 179°. In an actual application process, the jump affects calculation of an angle difference. Therefore, the jump needs to be corrected first.

In some exemplary embodiments, the motion signal of the user or feature information corresponding to the motion signal may also be analyzed by using a motion recognition model, to recognize the motion of the user. In some exemplary embodiments, the motion recognition model includes a machine learning model trained to recognize the motion of the user. In some exemplary embodiments, the motion recognition model may include one or more machine learning models. In some exemplary embodiments, the motion recognition model may include but is not limited to one or more of a machine learning model that classifies a user motion signal, a machine learning model that recognizes quality of a user motion, a machine learning model that recognizes a quantity of user motions, and a machine learning model that recognizes a degree of fatigue in performing a motion by the user.

FIG. 19 is an exemplary flowchart of a motion monitoring and feedback method according to some exemplary embodiments of this disclosure. As shown in FIG. 19, the process 1900 may include the following steps.

Step 1910: Obtain a motion signal of a moving user.

In some exemplary embodiments, this step may be performed by the obtaining module 210. In some exemplary embodiments, the motion signal includes at least feature information corresponding to a myoelectric signal and feature information corresponding to a posture signal. The motion signal refers to body parameter information during moving of the user. In some exemplary embodiments, the body parameter information may include but is not limited to one or more of a myoelectric signal, a posture signal, a heart rate signal, a temperature signal, a humidity signal, a blood oxygen concentration, and the like. In some exemplary embodiments, the motion signal may include at least a myoelectric signal and a posture signal. In some exemplary embodiments, a myoelectric sensor in the obtaining module 210 may capture a myoelectric signal during moving of the user, and a posture sensor in the obtaining module 210 may capture a posture signal during moving of the user.

Step 1920: Monitor a motion of the user based on the motion signal by using a motion recognition model, and perform motion feedback based on an output result of the motion recognition model.

In some exemplary embodiments, this step may be performed by the processing module 220 and/or the processing device 110. In some exemplary embodiments, the output result of the motion recognition model may include but is not limited to one or more of a motion type, motion quality, a quantity of motions, a fatigue index, and the like. For example, the motion recognition model may recognize the motion type of the user as seated chest fly based on the motion signal. As another example, one machine learning model in the motion recognition model may first recognize the motion type of the user as seated chest fly based on the motion signal, and another machine learning model in the motion recognition model may output the motion quality of the user motion as a standard motion or an erroneous motion based on the motion signal. In some exemplary embodiments, motion feedback may include sending out prompt information. In some exemplary embodiments, the prompt information may include but is not limited to a voice prompt, a text prompt, an image prompt, a video prompt, and the like. For example, the output result of the motion recognition model is an erroneous motion, and the processing device 110 may control the wearable device 130 or the mobile terminal device 140 to send out a voice prompt to the user (for example, information such as “non-standard motion”) to remind the user to adjust the motion in time. As another example, the output result of the motion recognition model is a standard motion, and the wearable device 130 or the mobile terminal device 140 may not send out prompt information, or send out prompt information similar to “standard motion”. In some exemplary embodiments, the motion feedback may also include that the wearable device 130 stimulates a corresponding moving part of the user. For example, an element of the wearable device 130 stimulates a corresponding motion part of the user through vibration feedback, electrical stimulation feedback, pressure feedback, or the like. For example, the output result of the motion recognition model is an erroneous motion, and the processing device 110 may control the element of the wearable device 130 to stimulate the corresponding moving part of the user. In some exemplary embodiments, motion feedback may further include outputting a motion record of during moving of the user. The motion record herein may refer to one or more of a user motion type, a moving duration, a quantity of motions, motion quality, a fatigue index, physiological parameter information during moving, and the like.

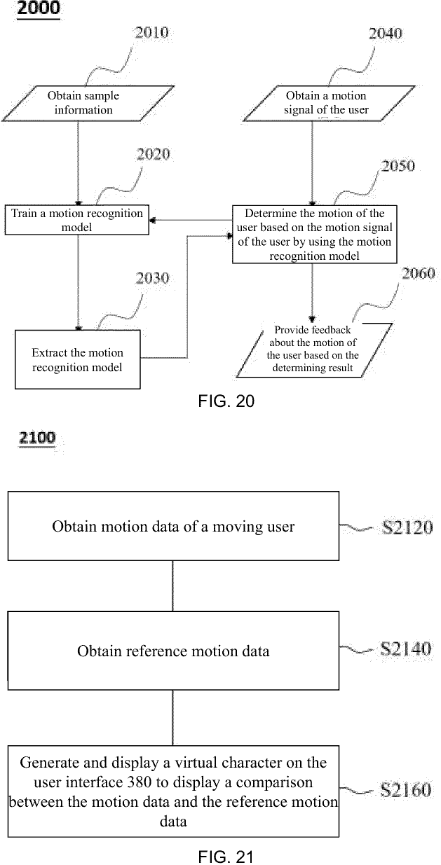

FIG. 20 is an exemplary flowchart of a model training application according to some exemplary embodiments of this disclosure. As shown in FIG. 20, the process 2000 may include the following steps.

Step 2010: Obtain sample information.

In some exemplary embodiments, this step may be performed by the obtaining module 210. In some exemplary embodiments, the sample information may include a motion signal during moving of a professional (for example, a fitness trainer) and/or a non-professional. For example, the sample information may include a myoelectric signal and/or a posture signal generated when a same type of motion (for example, seated chest fly) is performed by the professional and/or the non-professional. In some exemplary embodiments, the myoelectric signal and/or the posture signal in the sample information may be subjected to segmentation processing in the process 700, burr processing in the process 900, conversion processing in the process 1300, and the like, to form at least one myoelectric signal segment and/or posture signal segment. The at least one myoelectric signal segment and/or posture signal segment may be used as an input to a machine learning model to train the machine learning model. In some exemplary embodiments, feature information corresponding to the at least one myoelectric signal segment and/or feature information corresponding to the at least one posture signal segment may also be used as an input to the machine learning model to train the machine learning model. For example, frequency information and amplitude information of the myoelectric signal may be used as an input to the machine learning model. As another example, an angular velocity, a direction of the angular velocity, or an acceleration value of the angular velocity of the posture signal may be used an input to the machine learning model. As another example, a motion start point, a motion middle point, and a motion end point of the motion signal may be used as an input to the machine learning model. In some exemplary embodiments, the sample information may be obtained from a storage device of the processing device 110. In some exemplary embodiments, the sample information may be obtained from the obtaining module 210.

Step 2020: Train a motion recognition model.

This step may be performed by the processing device 110. In some exemplary embodiments, the motion recognition model may include one or more machine learning models. For example, the motion recognition model may include but is not limited to one or more of a machine learning model that classifies a user motion signal, a machine learning model that recognizes quality of a user motion, a machine learning model that recognizes a quantity of user motions, and a machine learning model that recognizes a degree of fatigue in performing a motion by a user.

In some exemplary embodiments, when training a machine learning model that recognizes a motion type of the user, sample information from different motion types (each myoelectric signal segment or posture signal segment) may be tagged. For example, sample information from a myoelectric signal and/or a posture signal generated when the user performs seated chest fly may be marked as “1”, where “1” is used to represent “seated chest fly”; and sample information from a myoelectric signal and/or a posture signal generated when the user performs biceps lifting may be marked as “2”, where “2” is used to represent “biceps lifting”. Feature information (for example, frequency information and amplitude information) of myoelectric signals and feature information (for example, angular velocities, directions of the angular velocities, and angular velocity values of the angular velocities) of posture signals corresponding to different motion types are different. By using tagged sample information (for example, feature information corresponding to a myoelectric signal and/or a posture signal in the sample information) as an input to the machine learning model to train the machine learning model, a motion recognition model for recognizing the motion type of the user can be obtained, and by inputting a motion signal to the machine learning model, a corresponding motion type can be output.

In some exemplary embodiments, the motion recognition model may further include a machine learning model for determining quality of a user motion. Sample information herein may include a standard motion signal (also referred to as a positive sample) and a non-standard motion signal (also referred to as a negative sample). The standard motion signal may include a motion signal generated when the professional performs a standard motion. In some exemplary embodiments, positive and negative samples in the sample information (each myoelectric signal segment or posture signal segment) may be tagged. For example, a positive sample is marked as “1” and a negative sample is marked as “0”. Herein, “1” is used to represent that the motion of the user is a standard motion, and “0” is used to represent that the motion of the user is an erroneous motion. The trained machine learning model may output different tags based on input sample information (for example, a positive sample and a negative sample). It should be noted that the motion recognition model may include one or more machine learning models for analyzing and recognizing quality of a user motion, and that different machine learning models may separately analyze and recognize sample information from different motion types.

In some exemplary embodiments, the motion recognition model may further include a model for recognizing a motion quantity of bodybuilding motions of the user. For example, segmentation processing in the process 700 is performed on a motion signal (for example, a myoelectric signal and/or a posture signal) in the sample information to obtain at least one group of a motion start point, a motion middle point, and a motion end point, and the motion start point, motion middle point, and motion end point in each group are respectively marked. For example, the motion start point is marked as 1, the motion middle point is marked as 2, and the motion end point is marked as 3. The marks are used as an input to the machine learning model, and by inputting a group of continuous “1”, “2”, and “3” to the machine learning model, one motion can be output. For example, by inputting three consecutive groups of “1”, “2”, and “3” to the machine learning model, three motions can be output.

In some exemplary embodiments, the motion recognition model may further include a machine learning model for recognizing a fatigue index of the user. The sample information herein may further include other physiological parameter signals such as an electrocardio signal, a respiratory frequency, a temperature signal, and a humidity signal. For example, different frequency ranges of electrocardio signals may be used as input data for the machine learning model, frequencies of electrocardio signals from 60 times/min to 100 times/min are marked as “1” (normal), and those less than 60 times/min or more than 100 times/min are marked as “2” (abnormal). In some exemplary embodiments, further segmentation may be performed based on an electrocardio signal frequency of the user, different indices are marked as input data, and the trained machine learning model may output a corresponding fatigue index based on the electrocardio signal frequency. In some exemplary embodiments, the machine learning model may be further trained with reference to physiological parameter signals such as a respiratory frequency and a temperature signal.

Step 2030: Extract the motion recognition model.

In some exemplary embodiments, this step may be performed by the processing device 110. In some exemplary embodiments, the processing device 110 and/or the processing module 220 may extract the motion recognition model. In some exemplary embodiments, the motion recognition model may be stored in the processing device 110, the processing module 220, or the mobile terminal device 140.

Step 2040: Obtain a motion signal of the user.

In some exemplary embodiments, this step may be performed by the obtaining module 210. For example, in some exemplary embodiments, a myoelectric sensor in the obtaining module 210 may obtain a myoelectric signal of the user, and a posture sensor in the obtaining module 210 may capture a posture signal of the user. In some exemplary embodiments, the motion signal of the user may further include other physiological parameter signals such as an electrocardio signal, a respiratory signal, a temperature signal, and a humidity signal during moving of the user. In some exemplary embodiments, after the motion signal of the user is obtained, the motion signal (for example, the myoelectric signal and/or the posture signal) may be subjected to the segmentation processing in the process 700, the burr processing in the process 900, the conversion processing in the process 1300, and the like, to form at least one myoelectric signal segment and/or posture signal segment.

Step 2050: Determine the motion of the user based on the motion signal of the user by using the motion recognition model.

This step may be performed by the processing device 110 and/or the processing module 220. In some exemplary embodiments, the processing device 110 and/or the processing module 220 may determine the motion of the user based on the motion recognition model. For example, the machine learning model that classifies a user motion signal may use the user motion signal as input data to output a corresponding motion type. As another example, the machine learning model that recognizes quality of a user motion may use the user motion signal as input data to output quality of the motion (for example, a standard motion or an erroneous motion). As another example, the machine learning model that recognizes a fatigue index of the user in performing a motion may use the user motion signal (for example, an electrocardio signal) as input data to output the fatigue index of the user. In some exemplary embodiments, the user motion signal and a determining result (output) of the machine learning model may also be used as sample information for training the motion recognition model to train the motion recognition model to optimize related parameters of the motion recognition model.

Step 2060: Provide feedback about the motion of the user based on the determining result.

In some exemplary embodiments, this step may be performed by the wearable device 130 and/or the mobile terminal device 140. Further, the processing device 110 and/or the processing module 220 send/sends out a feedback instruction to the wearable device 130 and/or the mobile terminal 140 based on the determining result of the user motion, and the wearable device 130 and/or the mobile terminal device 140 provide/provides feedback to the user based on the feedback instruction. In some exemplary embodiments, the feedback may include sending prompt information (for example, text information, picture information, video information, voice information, or indicator information) and/or performing a corresponding motion (current stimulation, vibration, pressure change, heat change, or the like) to stimulate a body of the user. For example, when the user performs a sit-up motion, by monitoring a motion signal of the user, it is determined that force applied to a trapezius during moving of the user is excessive (that is, a motion of the head and neck during moving of the user is not standard), and in this case, the input/output module 260 (for example, a vibration prompter) in the wearable device 130 and the mobile terminal device 140 (for example, a smart watch or a smartphone) perform a corresponding feedback motion (for example, applying a vibration to a body part of the user, or sending out a voice prompt) to prompt the user to adjust a force-generating part in time. In some exemplary embodiments, the mobile terminal device 140 may output a corresponding motion record by monitoring a motion signal during moving of the user to determine a motion type, quality of the motion, and a quantity of motions during moving of the user, so that the user can know a moving status during moving of the user.

In some exemplary embodiments, when feedback is provided to the user, the feedback may match user perception. For example, when a user motion is not standard, a vibration stimulation may be applied to an area corresponding to the user motion, and the user can know, based on the vibration stimulation, that the motion is not standard, but the vibration stimulation is within a range acceptable to the user. Further, a matching model may be established based on a user motion signal and the user perception to find an optimal balance point between the user perception and the real feedback.

In some exemplary embodiments, the motion recognition model may be further trained based on the user motion signal. In some exemplary embodiments, the training of the motion recognition model based on the user motion signal may include evaluating the user motion signal to determine confidence of the user motion signal. A magnitude of the confidence may indicate quality of the user motion signal. For example, the higher the confidence, the better the quality of the user motion signal. In some exemplary embodiments, evaluating the user motion signal may be performed at various stages such as motion signal capture, preprocessing, segmentation, and/or recognition.

In some exemplary embodiments, the training of the motion recognition model based on the user motion signal may further include: determining whether the confidence is greater than a confidence threshold (for example, 80), and if the confidence is greater than or equal to the confidence threshold, training the motion recognition model based on the user motion signal corresponding to the confidence as sample data; or if the confidence is less than the confidence threshold, training the motion recognition model without using the user motion signal corresponding to the confidence as sample data. In some exemplary embodiments, the confidence may include but is not limited to confidence of any one stage of motion signal capture, signal preprocessing, signal segmentation, signal recognition, and the like. For example, the confidence of the motion signal captured by the obtaining module 210 may be used as a determining criterion. In some exemplary embodiments, the confidence may alternatively be joint confidence of any several stages of motion signal capture, signal preprocessing, signal segmentation, signal recognition, and the like. The joint confidence may be calculated based on confidence of each stage and averaged or weighted thereof. In some exemplary embodiments, training the motion recognition model based on the user motion signal may be training in real time, periodically (for example, one day, one week, or one month), or for an amount of data satisfied.

In some exemplary embodiments, when the user motion is not standard, the processing device 110 and/or the processing module 220 send/sends a feedback instruction to the wearable device 130 and/or the mobile terminal 140 based on the determining result of the user motion, and the wearable device 130 and/or the mobile terminal device 140 provide/provides feedback to the user based on the feedback instruction, but the user feels well, resulting in a decrease of confidence of the user in the motion monitoring system 100. For example, when the user performs biceps lifting, a standard posture of the motion is shoulder relaxation, but the user thinks that the shoulder is relaxed; however, the shoulder generates force unconsciously, resulting in excessive force on a trapezius. In this case, the input/output module 260 (for example, a vibration prompter) in the wearable device 130 and the mobile terminal device 140 (for example, a smart watch or a smartphone) may perform a corresponding feedback(for example, applying a vibration to a body part of the user, or sending out a voice prompt) to prompt the user to adjust a force-generating part in time. In this case, the perception of the user may be contrary to an analysis result of the wearable device 130 and/or the mobile terminal device 140, and the feedback result of the wearable device 130 and/or the mobile terminal device 140 may be considered as inaccurate. Therefore, this disclosure further provides a motion data display method, which may compare motion data during moving of a user with reference motion data, display a comparison result on the user interface 380 on the mobile terminal device 140 by using an animation of a virtual character, and tell a user about meanings of various motion data and a basis for data analysis of the system to give suggestions, so that the user may visually observe a difference between a motion of the user and a reference motion by observing and comparing the animation of the virtual character on the user interface 380. In this way, a difference between an analysis result and user perception may be reduced, and the user may be promoted to realize that the motion of the user is indeed non-standard, accept the feedback from the motion monitoring system 100, quickly realize a problem existing in the motion of the user, and adjust the moving motion in time to perform scientific movement.

FIG. 21 is an exemplary flowchart of a motion data display method 2100 according to some exemplary embodiments of this disclosure. As described above, the motion data display system 160 may perform the motion data display method 2100 provided in this disclosure. Specifically, the motion data display system 160 may perform the motion data display method 2100 with the processing device 110, or may perform the motion data display method 2100 with the mobile terminal device 140. For ease of presentation, an example in which the motion data display system 160 performs the motion data display method 2100 on the mobile terminal device 140 is used for description. Specifically, the processor 320 in the mobile terminal device 140 may read an instruction set stored in its local storage medium, and then, as specified by the instruction set, perform the motion data display method 2100 provided in this disclosure. The motion data display method 2100 may include:

S2120. Obtain motion data of a moving user.

The motion data refers to body parameter information when the user completes motion of a motion type in a target posture. In some exemplary embodiments, the body parameter information may include but is not limited to one or more of a myoelectric signal, a posture signal, a heart rate signal, a temperature signal, a humidity signal, a blood oxygen concentration, and the like. The motion data may correspond to motion signals at a plurality of measurement positions on a body of the user. The motion data may be one or more motion signals at a plurality of measurement positions obtained by a sensor unit in the obtaining module 210 in the wearable device 130 during moving of the user. As described above, the sensor unit may include but is not limited to one or more of a myoelectric sensor, a posture sensor, an electrocardio sensor, a respiration sensor, a temperature sensor, a humidity sensor, an inertial sensor, a blood oxygen saturation sensor, a Hall sensor, a galvanic skin sensor, a rotation sensor, and the like. In some exemplary embodiments, the motion signal may include one or more of a myoelectric signal, a posture signal, an electrocardio signal, a respiratory frequency, a temperature signal, a humidity sensor, and the like.

In some exemplary embodiments, before step S2120, the user may send a motion data display request through the mobile terminal device 140. Specifically, the user may perform an operation through the user interface 380 on the mobile terminal device 140 to request to display motion data; the mobile terminal device 140 may receive an operation instruction of the user to generate the motion data display request, and send the motion data display request to the motion data display system 160; and upon receiving the motion data display request, the motion data display system 160 begins to perform the motion data display method 2100. For example, a button for triggering the motion data display request, such as “start recording”, “start displaying”, or “start comparison”, may be provided on the user interface 380. The user may trigger the button. The motion data may be a motion signal captured by the sensor unit of the obtaining module 210 within a time window after the motion data display system 160 receives the motion data display request, or may be a motion signal captured by the sensor unit of the obtaining module 210 within a time window before the motion data display system 160 receives the motion data display request.

The motion data may be a motion signal directly obtained by the sensor unit in the obtaining module 210, or may be a motion signal formed after signal processing procedures such as conventional filtering, rectification, and wavelet transformation, segmentation processing in the process 700, burr processing in the process 900, conversion processing in the process 1300, motion monitoring in the process 1900, and the like are performed on the motion signal directly obtained by the sensor unit in the obtaining module 210, or may be a signal obtained by permuting or combining any one or more of the foregoing processing procedures.

In some exemplary embodiments, the motion data may include a plurality of pieces of myoelectric data. In some exemplary embodiments, the myoelectric data may be myoelectric signals directly captured by a plurality of myoelectric sensors in the obtaining module 210 during moving of the user. In this case, the motion data display system 160 may obtain the plurality of pieces of myoelectric data from the obtaining module 210. In some exemplary embodiments, the myoelectric data may be myoelectric signals formed after signal processing procedures such as conventional filtering, rectification, and wavelet transformation, segmentation processing in the process 700, burr processing in the process 900, and motion monitoring in the process 1900 are performed on the myoelectric signals captured by the plurality of myoelectric sensors in the obtaining module 210 during moving of the user, or myoelectric signals formed by permuting or combining any one or more of the foregoing processing procedures. As described above, signal processing procedures such as conventional filtering, rectification, and wavelet transformation, segmentation processing in the process 700, burr processing in the process 900, and motion monitoring in the process 1900 may be performed by the processing module 220 and/or the processing device 110. Therefore, in this case, the motion data display system 160 may obtain the plurality of pieces of myoelectric data from the processing module 220 and/or the processing device 110. The myoelectric signal and feature information (for example, frequency information or amplitude information) corresponding to the myoelectric signal may reflect a status of a muscle during moving of the user. The myoelectric sensors may be placed in different positions of the wearable device 130 based on myoelectric signals to be obtained, to measure myoelectric signals corresponding to different muscle positions of the human body. For ease of presentation, measurement positions of a plurality of myoelectric sensors corresponding to the plurality of pieces of myoelectric data on the body of the user are defined as first measurement positions. The plurality of pieces of myoelectric data correspond to actual myoelectric signals of muscles at a plurality of first measurement positions on the body of the user during moving of the user. For ease of presentation, in the following description, an example in which the plurality of pieces of myoelectric data are myoelectric signals formed after signal processing procedures such as conventional filtering, rectification, and wavelet transformation, segmentation processing in the process 700, burr processing in the process 900, and motion monitoring in the process 1900 are performed on the myoelectric signals captured by the plurality of myoelectric sensors in the obtaining module 210 during moving of the user is used for description.

In some exemplary embodiments, the motion data may include a plurality of pieces of posture data. In some exemplary embodiments, the posture data may be posture signals directly captured by a plurality of posture sensors in the obtaining module 210 during moving of the user. In this case, the motion data display system 160 may obtain the plurality of pieces of posture data from the obtaining module 210. In some exemplary embodiments, the posture data may be posture signals formed after conventional filtering processing, segmentation processing in the process 700, burr processing in the process 900, conversion processing in the process 1300, and motion monitoring in the process 1900 are performed on the posture signals captured by the plurality of posture sensors in the obtaining module 210 during moving of the user, or posture signals formed by permuting or combining any one or more of the foregoing processing procedures. As described above, conventional filtering processing, segmentation processing in the process 700, burr processing in the process 900, conversion processing in the process 1300, and motion monitoring in the process 1900 may be performed by the processing module 220 and/or the processing device 110. Therefore, in this case, the motion data display system 160 may obtain the plurality of pieces of posture data from the processing module 220 and/or the processing device 110. The posture signal and feature information (for example, a direction of an angular velocity, an angular velocity value, an acceleration value of the angular velocity, an angle, displacement information, and stress) corresponding to the posture signal may reflect a posture during moving of the user. The posture sensors may be placed at different positions of the wearable device 130 (for example, positions corresponding to a torso, four limbs, and joints, in the wearable device 130) based on the posture signals to be obtained, to measure posture signals corresponding to different positions on the human body. For ease of presentation, measurement positions of a plurality of posture sensors corresponding to the plurality of pieces of posture data on the body of the user are defined as second measurement positions. The plurality of pieces of posture data correspond to actual postures at the plurality of second measurement positions on the body of the user during moving of the user. For ease of presentation, in the following description, an example in which the plurality of pieces of posture data are posture signals formed after conventional filtering processing, segmentation processing in the process 700, burr processing in the process 900, conversion processing in the process 1300, and motion monitoring in the process 1900 are performed on the posture signals captured by the plurality of posture sensors in the obtaining module 210 during moving of the user is used for description.

In some exemplary embodiments, the motion data may include both the plurality of pieces of myoelectric data and the plurality of pieces of posture data.

As described above, the processing module 220 and/or the processing device 110 may perform the segmentation processing on the motion signal in the process 700. The processing module 220 and/or the processing device 110 may segment the motion signal based on at least one target feature in the motion signal. In some exemplary embodiments, the target feature may include one or more of a motion start point, a motion middle point, or a motion end point. A motion signal between a motion start point and a next motion start point may be considered as a motion signal segment, that is, a full motion period. In some exemplary embodiments, the motion data may include at least one full motion period. In some exemplary embodiments, the motion data may include the at least one target feature.

As described above, in the process 600 and the process 1900, the processing module 220 and/or the processing device 110 may monitor the motion during moving of the user based on at least one motion signal segment. Specifically, the processing module 220 and/or the processing device 110 may match the at least one motion signal segment with at least one preset motion signal segment based on a motion recognition model, and may determine a motion type of the user by determining a matching degree between the at least one motion signal segment and the at least one preset motion signal segment. The motion type refers to a bodybuilding motion taken during moving of the user. In some exemplary embodiments, the motion type may include but is not limited to one or more of seated chest fly, deep squat exercise, hard pull exercise, plank exercise, running, swimming, and the like. In some exemplary embodiments, the motion data may further include motion type data during moving of the user. The motion type data may be an identity corresponding to the motion type. Different motion types correspond to different identities. For example, “1” may be used to represent “seated chest fly”, and “2” may be used to represent “biceps lifting”.

The motion data display method 2100 may further include:

S2140. Obtain reference motion data.

As described above, standard motion signals corresponding to different motions may be preset in the database and/or the processing device 110. The standard motion signals corresponding to the different motions may be motion signals when a professional (for example, a fitness trainer) moves. The standard motion signals corresponding to the different motions may be motion signals when one professional moves, or may be an integrated signal after fusion processing is performed on motion signals when a plurality of professionals move. The fusion processing includes but is not limited to fusion methods such as mean fusion and principal component analysis. The standard motion signals corresponding to the different motions may be myoelectric signals and/or posture signals when a professional (for example, a fitness trainer) moves. The standard motion signals corresponding to the different motions may be myoelectric signals and/or posture signals generated when a professional (for example, a fitness trainer) moves, and directly obtained from the sensor unit in the obtaining module 210 in the wearable device 130. In some exemplary embodiments, myoelectric signals and/or posture signals in the standard motion signals corresponding to the different motions may be at least one myoelectric signal segment and/or posture signal segment in a full motion period, formed after segmentation processing in the process 700, burr processing in the process 900, conversion processing in the process 1300, and the like are performed on the myoelectric signals and/or posture signals that are generated during moving of a professional (for example, a fitness trainer) and directly obtained from the sensor unit in the obtaining module 210 in the wearable device 130. In some exemplary embodiments, the reference motion data may include at least one target feature of the reference motion data.

In some exemplary embodiments, the standard motion signals corresponding to the different motions may be stored in the processing device 110. In some exemplary embodiments, the standard motion signals corresponding to the different motions may alternatively be stored in the processing module 220 of the wearable device 130. In some exemplary embodiments, the standard motion signals corresponding to the different motions may alternatively be stored in the mobile terminal device 140. In some exemplary embodiments, the standard motion signals corresponding to the different motions may alternatively be stored in the database.

The reference motion data corresponds to the motion data. In other words, the reference motion data may be data related to a standard motion signal matching the motion type of the user. In some exemplary embodiments, the motion data display system 160 may obtain the reference motion data corresponding to the motion data directly from a device storing standard signals corresponding to different motions. In some exemplary embodiments, the motion data display system 160 may find and obtain, based on the motion type data during moving of the user in the motion data, the reference motion data corresponding to the motion data from the device storing standard signals corresponding to different motions.

In some exemplary embodiments, the motion data may include the plurality of pieces of myoelectric data. Correspondingly, the reference motion data may include a plurality of pieces of reference myoelectric data corresponding to the plurality of pieces of myoelectric data. The plurality of pieces of reference myoelectric data correspond to reference myoelectric signals at the plurality of first measurement positions when the user performs moving of the motion type, that is, standard myoelectric signals generated by standard motions corresponding to the motion type.

In some exemplary embodiments, the motion data may include the plurality of pieces of posture data. Correspondingly, the reference motion data may include a plurality of pieces of reference posture data corresponding to the plurality of pieces of posture data. The plurality of pieces of reference posture data correspond to reference posture signals at the plurality of second measurement positions when the user performs of the motion of the motion type, that is, standard posture signals generated by standard motions corresponding to the motion type.

The motion data display method 2100 may further include:

S2160. Generate and display a virtual character on the user interface 380 to display a comparison between the motion data and the reference motion data.

The virtual character may be a model matching the user. The virtual character may be a preset three-dimensional or two-dimensional animation model. In some exemplary embodiments, the virtual character may include a virtual user character and a virtual reference character. The virtual user character may be associated with the motion data, that is, the motion data may be combined with the virtual user character to display the motion data through the virtual user character. The virtual reference character may be associated with the reference motion data, that is, the reference motion data may be combined with the virtual reference character to display the reference motion data through the virtual reference character. In some exemplary embodiments, the virtual user character and the virtual reference character may be represented as two independent virtual characters. In some exemplary embodiments, the virtual user character and the virtual reference character may be represented as the same virtual character. The virtual user character and the virtual reference character may use a same animation model or may use different animation models. For ease of presentation, in the following description, an example in which the virtual user character and the virtual reference character are the same animation model is used for presentation.

The motion data display system 160 may combine the motion data and the reference motion data with the virtual character to display the comparison between the motion data and the reference motion data to feed back an evaluation result of the motion data to the user in a more intuitive manner.

As described above, in some exemplary embodiments, the motion data may include the plurality of pieces of myoelectric data. Correspondingly, the reference motion data may include the plurality of pieces of reference myoelectric data. In this case, step S2160 may include generating and displaying a myoelectric animation of the virtual character on the user interface 380 to display the comparison between the motion data and the reference motion data. An animation of the virtual character may include the myoelectric animation of the virtual character.

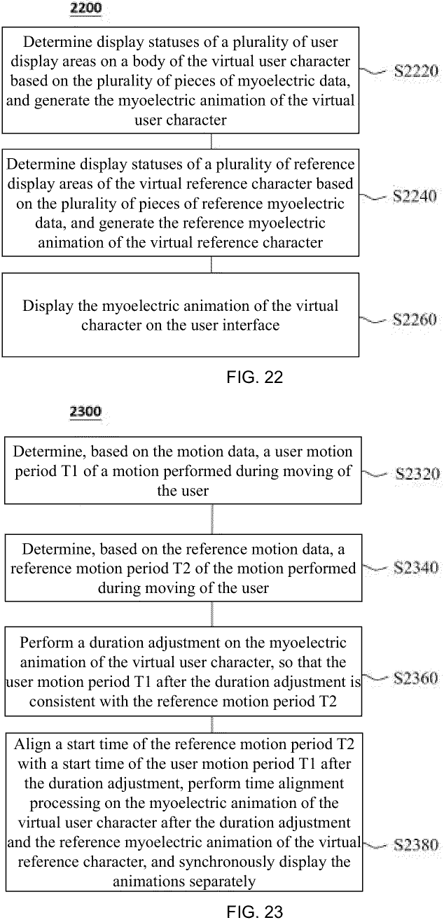

In some exemplary embodiments, the virtual user character and the virtual reference character may be represented as two independent virtual characters. In some exemplary embodiments, the virtual user character and the virtual reference character may be represented as the same virtual character. When the virtual user character and the virtual reference character may be represented as two independent virtual characters, the myoelectric animation of the virtual character may include a myoelectric animation of the virtual user character and a reference myoelectric animation of the virtual reference character. FIG. 22 is an exemplary flowchart for generating a myoelectric animation according to some exemplary embodiments of this disclosure. As shown in FIG. 22, the process 2200 may include:

S2220. Determine display statuses of a plurality of user display areas on a body of the virtual user character based on the plurality of pieces of myoelectric data, and generate the myoelectric animation of the virtual user character.

The plurality of user display areas correspond to the plurality of first measurement positions. The user display area may be an area on the body of the virtual user character and corresponding to a muscle group measured by the myoelectric data. For example, a myoelectric sensor for measuring a pectoralis major may be located at a position of the pectoralis major of the user, and a user display area on the body of the virtual user character corresponding to the position is also the display area corresponding to the position of the pectoralis major. For example, a myoelectric sensor for measuring a biceps brachii may be located at a position of the biceps brachii of the user, and a user display area on the body of the virtual user character corresponding to the position is also the display area corresponding to the position of the biceps brachii. Each user display area may correspond to one or more of the plurality of first measurement positions. The user display area may correspond to a large muscle group on the body of the virtual user character or may correspond to a small muscle group on the body of the virtual user character. In some exemplary embodiments, one muscle group may be provided with one myoelectric sensor. In some exemplary embodiments, one muscle group may be provided with a plurality of myoelectric sensors. Different muscle groups may be provided with different quantities of myoelectric sensors based on conditions of the muscle groups. For example, the shoulder may be provided with a plurality of myoelectric sensors to measure myoelectric signals of an anterior deltoid, a posterior deltoid, and a middle deltoid respectively.

Specifically, step S2220 may include: for each of the plurality of user display areas on the body of the virtual user character, determining, based on myoelectric data at a first measurement position corresponding to the user display area, a user force contribution ratio of a muscle at the first measurement position corresponding to the user display area to a motion during moving of the user; and determining a display status of each user display area based on the user force contribution ratio corresponding to the user display area. The force contribution may be a contribution of a muscle measured by the current myoelectric data at a current time to completion of moving by the user during moving of the user. The force contribution ratio may be a proportion of the current myoelectric data to the plurality of pieces of myoelectric data, that is, a proportion of force generated by the muscle corresponding to the current myoelectric data to force generated by all muscles. The force contribution ratio may be a ratio of an amplitude level of force generated by the muscle at the current time, as measured by the current myoelectric data, to an amplitude level of maximum force generated by the muscle. The force contribution ratio may alternatively be a ratio of an amplitude level of force generated by the muscle at the current time, as measured by the current myoelectric data, to a statistical value (mean, median, or the like) of the muscle at the current time among a plurality of normal users. The force contribution ratio may alternatively be a ratio of a myoelectric intensity of the muscle measured by the current myoelectric data to a sum of myoelectric intensities of all muscles. The force contribution ratio may alternatively be a ratio of a product of a myoelectric intensity of the muscle measured by the current myoelectric data and a muscle volume to a sum of all products of myoelectric intensities and muscle volumes of all muscles. The force contribution ratio is obtained based on the myoelectric data after data processing. The data processing method may be filtering and rectifying a myoelectric amplitude level in the enveloped myoelectric data. The data processing method may alternatively include extracting a feature amplitude after performing a wavelet transform, a Fourier transform, or the like on the myoelectric data. The data processing method may alternatively be performing arbitrary processing on the myoelectric data. The display status of the user display area and the user force contribution ratio of the muscle at the first measurement position corresponding to the user display area are displayed based on a preset relationship. For example, the display status of the user display area may be represented by a difference in display colors or a difference in filling densities. For ease of presentation, an example in which the display status of the user display area is represented by the difference in display colors is used for description. For example, a muscle energy diagram is used to visually display a change in muscle energy during moving of the user, and a current magnitude of force generated by the muscle is displayed by changing a display color of the user display area. For example, the smaller the user force contribution ratio of the muscle, the lighter the color of the corresponding user display area, or the larger the user force contribution ratio of the muscle, the darker the color of the corresponding user display area. For example, the display color of the user display area may be changed from green, yellow, red, crimson, purple, and black based on the user force contribution ratios in ascending order. The color change is used as an example only, and a person of ordinary skill in the art should know that the change of the display status of the user display area is not limited to the color change in this disclosure. The user display area may be composed of a plurality of different colors, and the display color is changed based on user force contribution ratios corresponding to the colors. Each different color of the user display area may correspond to one user force contribution ratio range. The display color of the user display area may be determined based on a range in which the user force contribution ratio is located.

As described above, the motion data may include motion data of at least one full motion period. When the user moves, force-generating positions of muscles and force contribution ratios may be different at different times in a full motion period. Therefore, the display status of the same user display area may change due to the change of the myoelectric data. A change of the display status of each user display area may be determined based on the changes of the plurality of pieces of myoelectric data, so that the myoelectric animation of the virtual user character is generated. The myoelectric animation of the virtual user character may include a plurality of image frames. In some exemplary embodiments, myoelectric data captured by each myoelectric sensor at a time may form one image frame. In some exemplary embodiments, more image frames may be generated by an interpolation algorithm between adjacent myoelectric data captured by each myoelectric sensor. In some exemplary embodiments, myoelectric data captured by each myoelectric sensor at a plurality of times may form one image frame. In some exemplary embodiments, the motion data display system 160 may select myoelectric data at a plurality of time points based on a time-varying curve of each piece of myoelectric data, determine the display status of each user display area based on the myoelectric data, and generate a plurality of image frames, thereby generating the myoelectric animation of the virtual user character.

In some exemplary embodiments, the myoelectric animation of the virtual character may include a plurality of animations. For example, the myoelectric animation of the virtual character may include both a front myoelectric animation and a back myoelectric animation when there are many muscle groups mobilized during moving of the user, including both a front muscle group and a back muscle group.

As shown in FIG. 22, the process 2200 may further include:

S2240. Determine display statuses of a plurality of reference display areas of the virtual reference character based on the plurality of pieces of reference myoelectric data, and generate the reference myoelectric animation of the virtual reference character.

The plurality of reference display areas correspond to the plurality of first measurement positions. The plurality of pieces of reference myoelectric data corresponds to the plurality of pieces of myoelectric data. To be specific, there is a one-to-one correspondence between the plurality of reference myoelectric data and the plurality of pieces of myoelectric data, and there is also a one-to-one correspondence between the measured first measurement positions thereof. Therefore, the correspondence between the plurality of pieces of reference myoelectric data and the plurality of first measurement positions is consistent with the correspondence between the plurality of pieces of myoelectric data and the plurality of first measurement positions. Details will not be described herein again.

Specifically, step S2240 may include: for each of the plurality of reference display areas on a body of the virtual reference character, determining, based on reference myoelectric data at a first measurement position corresponding to the reference display area, a reference force contribution ratio of a muscle at the first measurement position corresponding to the reference display area to a motion during moving of the user; and determining a display status of each reference display area based on the reference force contribution ratio corresponding to the reference display area. Step S2240 is basically consistent with step S2220 and will not be described herein again.

The display status of the reference display area and the reference force contribution ratio of the muscle at the first measurement position corresponding to the reference display area may be displayed based on a preset relationship. The relationship between the display status of the reference display area and the reference force contribution ratio of the muscle at the first measurement position corresponding to the reference display area is consistent with the relationship between the display status of the user display area and the user force contribution ratio of the muscle at the first measurement position corresponding to the user display area, and is not described herein again.

As shown in FIG. 22, the process 2200 may further include:

S2260. Display the myoelectric animation of the virtual character on the user interface 380.

In the case where the virtual user character and the virtual reference character are represented as two independent virtual characters, displaying the myoelectric animation of the virtual character on the user interface 380 may be displaying the myoelectric animation of the virtual user character and the reference myoelectric animation of the virtual reference character on the user interface 380 separately. For example, the myoelectric animation of the virtual user character is displayed in an upper half of the user interface 380, and the reference myoelectric animation of the virtual reference character is displayed in a lower half of the user interface 380. Alternatively, the myoelectric animation of the virtual user character may be displayed in a left half of the user interface 380, and the reference myoelectric animation of the virtual reference character is displayed may be a right half of the user interface 380.

In some exemplary embodiments, displaying the myoelectric animation of the virtual character on the user interface 380 may be displaying the myoelectric animation of the virtual user character and the reference myoelectric animation of the virtual reference character directly on the user interface 380 separately. In some exemplary embodiments, because there is a difference between a motion period of moving by the user and a motion period of a standard motion, to determine a difference between the myoelectric data and the reference myoelectric data in the same posture more conveniently and visually, the motion data display system 160 may perform a duration adjustment on the user motion period corresponding to the myoelectric data when the user performs the motion of the motion type or the reference motion period of the standard motion corresponding to the reference myoelectric data, so that synchronous displaying may be performed separately after the user motion period and the reference motion period become consistent.

FIG. 23 is an exemplary flowchart for separately displaying myoelectric animations according to some exemplary embodiments of this disclosure. As shown in FIG. 23, the process 2300 may include the following steps.

S2320. Determine, based on the motion data, a user motion period T1 of a motion performed by the user.

As described above, the processing module 220 and/or the processing device 110 may perform the segmentation processing on the motion signal in the process 700. The processing module 220 and/or the processing device 110 may segment the motion signal based on at least one target feature in the motion signal. In some exemplary embodiments, the target feature may include one or more of a motion start point, a motion middle point, or a motion end point. A motion signal between a motion start point and a next motion start point may be considered as a motion signal segment, that is, a full motion period. In some exemplary embodiments, the motion data may include at least one full motion period. In some exemplary embodiments, the motion data may include the at least one target feature. The motion data display system 160 may directly determine the user motion period T1 of the motion data based on the motion data, or may determine the user motion period T1 based on at least one target feature in the motion data, such as one or more of a motion start point, a motion middle point, and a motion end point.

S2340. Determine, based on the reference motion data, a reference motion period T2 of the motion performed by the user.

As described above, the reference motion data includes at least one full motion period. In some exemplary embodiments, the reference motion data may include at least one target feature. The motion data display system 160 may directly determine the reference motion period T2 of the reference motion data based on the reference motion data, or may determine the reference motion period T2 based at least one target feature in the reference motion data, such as one or more of a motion start point, a motion middle point, and a motion end point.

S2360. Perform a duration adjustment on the myoelectric animation of the virtual user character, so that the user motion period T1 after the duration adjustment is consistent with the reference motion period T2.

In some exemplary embodiments, the motion data display system 160 may use the reference motion period T2 as a reference to perform a duration adjustment on the user motion period T1, so that the user motion period T1 after the duration adjustment is consistent with the reference motion period T2. In some exemplary embodiments, the motion data display system 160 may use the user motion period T1 as a reference to perform a duration adjustment on the reference motion period T2, so that the reference motion period T2 after the duration adjustment is consistent with the user motion period T1. For ease of presentation, an example in which the motion data display system 160 may use the reference motion period T2 as a reference to perform a duration adjustment on the user motion period T1, so that the user motion period T11 after the duration adjustment is consistent with the reference motion period T2, is used for description. A manner of performing a duration adjustment on the user motion period T1 may be linear scaling or non-linear scaling, or may be automatic scaling by using a trajectory matching algorithm (such as a DTW algorithm).

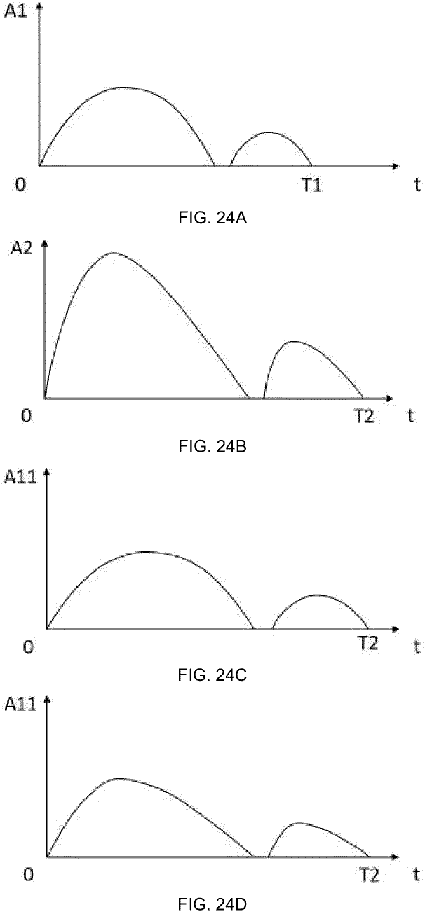

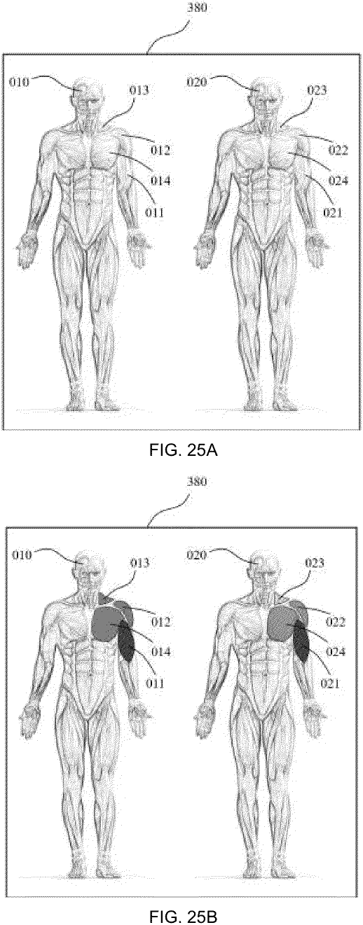

An example in which the motion type of the user is biceps lifting is used for description. FIG. 24A is a time-varying curve of myoelectric data of the biceps brachii according to some exemplary embodiments of this disclosure. A horizontal axis is a time t and a vertical axis is an amplitude A1. As shown in FIG. 24A, a time t = 0 is a motion start point, and a time t = T1 is a motion end point of motion data of the user. FIG. 24B is a time-varying curve of reference myoelectric data of the biceps brachii according to some exemplary embodiments of this disclosure. A horizontal axis is a time t and a vertical axis is an amplitude A2. As shown in FIG. 24B, a time t = 0 is a motion start point, and a time t = T2 is a motion end point of reference motion data. FIG. 24C is a time-varying curve of myoelectric data of the biceps brachii after a linear duration adjustment according to some exemplary embodiments of this disclosure. A horizontal axis is a time t and a vertical axis is an amplitude A11. As shown in FIG. 24C, the linear duration adjustment may be a uniformly scaled duration adjustment on the user motion period T1, so that the amplitude A11(t) at the time t is equal to A1 (tT1/T2). FIG. 24D is a time-varying curve of myoelectric data of the biceps brachii after a non-linear duration adjustment according to some exemplary embodiments of this disclosure. A horizontal axis is a time t and a vertical axis is an amplitude A11. As shown in FIG. 24D, the non-linear duration adjustment may be scaling the user motion period T1 based on change features of the reference motion data and the motion data. For example, a time corresponding to a maximum amplitude in the motion data is made consistent with a time corresponding to a maximum amplitude in the reference motion data, and a time corresponding to a minimum amplitude in the motion data is made consistent with a time corresponding to a minimum amplitude in the reference motion data.

As shown in FIG. 23, the process 2300 may further include:

S2380. Align a start time of the reference motion period T2 with a start time of the user motion period T1 after the duration adjustment, perform time alignment processing on the myoelectric animation of the virtual user character after the duration adjustment and the reference myoelectric animation of the virtual reference character, and synchronously display the animations separately.

In step S2380, the motion data display system 160 may adjust the myoelectric animation of the virtual user character based on the duration adjustment rule, so that the myoelectric animation of the virtual user character is consistent with the reference motion data after the duration adjustment. When displaying the myoelectric animation, the motion data display system 160 may align the start time of the myoelectric animation of the virtual user character after the duration adjustment with the start time of the reference myoelectric animation of the virtual reference character, so that the myoelectric animation of the virtual user character after the duration adjustment and the reference myoelectric animation of the virtual reference character start to be synchronously played from the same motion point, so as to compare the myoelectric animation of the virtual user character with the reference myoelectric animation of the virtual reference character more visually, display a difference between the motion data and the reference motion data more visually, and help the user move scientifically.

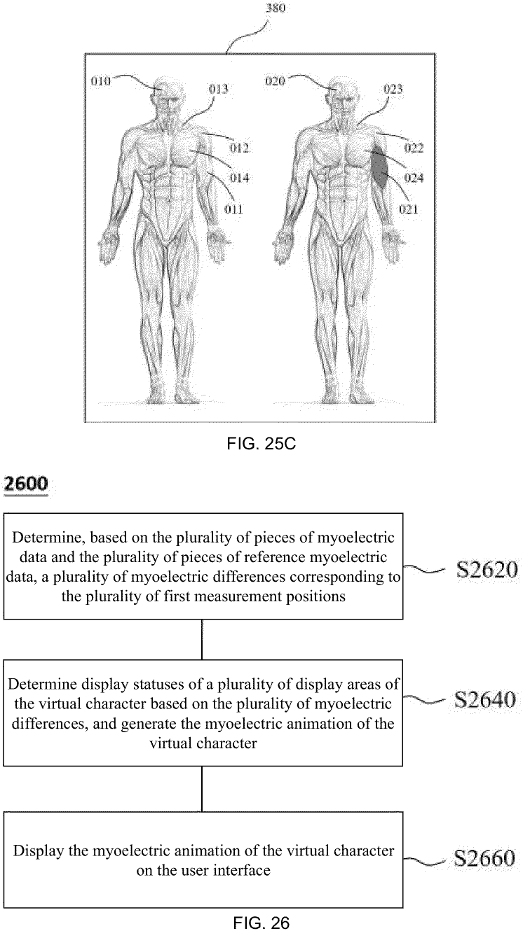

For ease of presentation, a motion of biceps lifting performed by a left arm is used as an example for description. In the motion of biceps lifting, main muscles that participate in moving include a biceps, a deltoid, a trapezius, and a pectoral muscle. FIG. 25A to FIG. 25C are schematic diagrams of the user interface 380 according to some exemplary embodiments of this disclosure. In FIG. 25A to FIG. 25C the myoelectric animation 010 of the virtual user character and the reference myoelectric animation 020 of the virtual reference character may be displayed separately on the user interface 380. For example, in FIG. 25A to FIG. 25C, the myoelectric animation 010 of the virtual user character is displayed in the left half of the user interface 380 and the reference myoelectric animation 020 of the virtual reference character is displayed in the right half of the user interface 380. The user interface 380 shown in FIG. 25A corresponds to a myoelectric animation at a time before the motion starts. As shown in FIG. 25A, both the motion data of the user and the reference motion data of the standard motion are in a relaxed state before the motion starts, and therefore, no force is generated by the muscles. In this case, none of a user display area 011 corresponding to the biceps brachii, a user display area 012 corresponding to the deltoid, a user display area 013 corresponding to the trapezius, and a user display area 014 corresponding to the pectoral muscle in the myoelectric animation 010 of the virtual user character is displayed in color; and none of a user display area 021 corresponding to the biceps brachii, a user display area 022 corresponding to the deltoid, a user display area 023 corresponding to the trapezius, and a user display area 024 corresponding to the pectoral muscle in the reference myoelectric animation 020 of the virtual reference character is displayed in color.

The user interface 380 shown in FIG. 25B corresponds to a myoelectric animation at a time during the motion of biceps lifting. During the motion of biceps lifting, a main force-generating point should theoretically be the biceps brachii, and in some cases, the pectoral muscle may generate force slightly, for example, when the user does not keep the chin up and the chest out. In a standard motion of biceps lifting, the trapezius should not or seldom participate in generating force. As shown in FIG. 25B, the display color of the user display area 013 corresponding to the trapezius in the myoelectric animation 010 of the virtual user character is darker than the color of the reference display area 023 corresponding to the trapezius in the reference myoelectric animation 020 of the virtual reference character, indicating that when the user performs biceps lifting, the trapezius generates relatively great force greater than the force generated by the trapezius in the standard motion of biceps lifting.

The user interface 380 shown in FIG. 25C corresponds to a myoelectric animation from the end of the motion of biceps lifting to a time before the start of a next motion period. In a group of consecutive motions of biceps lifting, it is impossible to relax completely from the end of one full motion period to the start of a next full motion period. In other words, when a barbell reaches the bottom, the biceps brachii cannot relax completely, but should keep generating force to achieve a best exercise effect. As shown in FIG. 25C, in the myoelectric animation 010 of the virtual user character, no color is displayed in the user display area 011 corresponding to the biceps brachii, and it can be seen that the user is in a fully relaxed state. However, the reference myoelectric animation 020 of the virtual reference character has a darker color in the reference display area 021 corresponding to the biceps brachii.

In summary, by observing the myoelectric animation 010 of the virtual user character and the reference myoelectric animation 020 of the virtual reference character, the user may clearly and visually see the difference between the muscle energy diagram in the myoelectric animation 010 of the virtual user character and the standard muscle energy diagram in the reference myoelectric animation 020 of the virtual reference character, find a problem existing in the current moving motion, and perform an adjustment in time.

As described above, in some exemplary embodiments, the virtual user character and the virtual reference character may be displayed as the same virtual character on the user interface 380. When the virtual user character and the virtual reference character are represented as the same virtual character, the animation of the virtual character may include a myoelectric animation generated based on the difference between the motion data and the reference motion data. FIG. 26 is an exemplary flowchart for generating a myoelectric animation according to other embodiments of this disclosure. As shown in FIG. 26, the process 2600 may include:

S2620. Determine, based on the plurality of pieces of myoelectric data and the plurality of pieces of reference myoelectric data, a plurality of myoelectric differences corresponding to the plurality of first measurement positions.

As described above, the plurality of pieces of myoelectric data correspond to the plurality of pieces of reference myoelectric data. Specifically, there is a one-to-one correspondence between the plurality of pieces of myoelectric data and the plurality of pieces of reference myoelectric data. Each piece of myoelectric data and reference myoelectric data corresponding to the myoelectric data measure an actual myoelectric signal and a standard myoelectric signal of a muscle at the same position during moving of the user. When the virtual user character and the virtual reference character are represented as the same virtual character, to display the comparison between the myoelectric data and the reference myoelectric data, the animation of the virtual character may be an animation formed based on the difference between the myoelectric data and the reference myoelectric data.

Specifically, each of the plurality of first measurement positions corresponds to myoelectric data and reference myoelectric data. The motion data display system 160 may determine, based on myoelectric data corresponding to a current first measurement position, a user force contribution ratio of a muscle corresponding to the first measurement position to a motion during moving of the user; determine, based on reference myoelectric data corresponding to the first measurement position, a reference force contribution ratio of the muscle corresponding to the first measurement position to the motion during moving of the user; and determine, based on the user force contribution ratio and the reference force contribution ratio corresponding to the first measurement position, a myoelectric difference corresponding to the first measurement position.

The force contribution ratio may be a contribution of a muscle measured by the current myoelectric data at the first measurement position at the current time to completion of moving by the user during moving of the user. The force contribution ratio may be a proportion of the current myoelectric data to the plurality of pieces of myoelectric data, that is, a proportion of force generated by the muscle corresponding to the current myoelectric data to force generated by all muscles. The force contribution ratio may be a ratio of an amplitude level of force generated by the muscle at the current time, as measured by the current myoelectric data, to an amplitude level of maximum force generated by the muscle. The force contribution ratio may alternatively be a ratio of an amplitude level of force generated by the muscle at the current time, as measured by the current myoelectric data, to a statistical value (mean, median, or the like) of the muscle at the current time among a plurality of normal users. The force contribution ratio may alternatively be a ratio of a myoelectric intensity of the muscle measured by the current myoelectric data to a sum of myoelectric intensities of all muscles. The force contribution ratio may alternatively be a ratio of a product of a myoelectric intensity of the muscle measured by the current myoelectric data and a muscle volume to a sum of all products of myoelectric intensities and muscle volumes of all muscles. The force contribution ratio is obtained based on the myoelectric data after data processing. The data processing method may be filtering and rectifying a myoelectric amplitude level in the enveloped myoelectric data. The data processing method may alternatively be a feature amplitude extracted after performing a wavelet transform, a Fourier transform, or the like on the myoelectric data. The data processing method may alternatively be performing arbitrary processing on the myoelectric data. The user force contribution ratio may be a contribution of a muscle corresponding to the current first measurement position to completion of moving by the user during actual moving of the user. The reference force contribution ratio may be a contribution of a muscle corresponding to the current first measurement position to completion of moving during moving of a professional (for example, a trainer). By calculating a difference between the user force contribution ratio and the reference force contribution ratio, a difference between the myoelectric data during moving of the user and reference myoelectric data during moving of the professional may be obtained.

As described above, in some exemplary embodiments, the motion period of moving by the user may differ from the motion period of the standard motion. To determine the difference between the myoelectric data and the reference myoelectric data in the same posture more conveniently and visually, the motion data display system 160 may perform a duration adjustment on the user motion period corresponding to the myoelectric data or the reference motion period of the standard motion corresponding to the reference myoelectric data when the user performs the motion of the motion type, and determine a plurality of myoelectric differences corresponding to the plurality of first measurement positions after the user motion period is consistent with the reference motion period.

Specifically, the motion data display system 160 may determine the user motion period T1 based on the motion data, as shown in step S2320. The motion data display system 160 may determine the reference motion period T2 based on the reference motion data, as shown in step S2340. In some exemplary embodiments, the motion data display system 160 may use the reference motion period T2 as a reference to perform a duration adjustment on the user motion period T1, so that the user motion period T1 after the duration adjustment is consistent with the reference motion period T2. In some exemplary embodiments, the motion data display system 160 may use the user motion period T1 as a reference to perform a duration adjustment on the reference motion period T2, so that the reference motion period T2 after the duration adjustment is consistent with the user motion period T1. For ease of presentation, an example in which the motion data display system 160 may use the reference motion period T2 as a reference to perform a duration adjustment on the user motion period T1, so that the user motion period T1 after the duration adjustment is consistent with the reference motion period T2, is used for description, as shown in step S2360. The motion data display system 160 may select several pieces of data at the same time from the myoelectric data and the reference myoelectric data after the duration adjustment processing, and perform step S2620 to obtain the myoelectric difference.

As shown in FIG. 26, the process 2600 may further include:

S2640. Determine display statuses of a plurality of display areas of the virtual character based on the plurality of myoelectric differences, and generate the myoelectric animation of the virtual character.

The correspondence between the plurality of display areas and the plurality of first measurement positions is consistent with the foregoing correspondence between the plurality of user display areas and the plurality of first measurement positions, and will not be described herein again.

The display status of the display area and the myoelectric difference of the first measurement position corresponding to the display area may be displayed based on a preset relationship. For example, the display status of the display area may be represented by a difference in display colors or a difference in filling densities. For ease of presentation, an example in which the display status of the display area is represented by a difference in display colors is used for description. For example, a force generation difference between the user corresponding to the current muscle and a professional is displayed by changing the display color of the display area. For example, the smaller the difference between the user and the professional, the lighter the color of the corresponding display area, or the larger the difference between the user and the professional, the darker the color of the corresponding user display area. As another example, when the force contribution ratio of the user exceeds the force contribution ratio of the professional, the display color of the display area is a cold tone, or when the force contribution ratio of the user is lower than the force contribution ratio of the professional, the display color of the display area is a warm tone. For example, when the force contribution ratio of the user exceeds the force contribution ratio of the professional, the display color of the display area may change from light green to dark green or from light blue to dark blue in ascending order of myoelectric differences. When the force contribution ratio of the user is lower than the force contribution ratio of the professional, the display color of the display area may change from yellow or orange to red or crimson in ascending order of myoelectric differences. Each different color of the display area may correspond to a difference myoelectric range. The display color of the display area may be determined based on a range in which the myoelectric difference is located.

As described above, the motion data may include motion data of at least one full motion period. When the user moves, force-generating positions of muscles and force contribution ratios may be different at different times in a full motion period. Therefore, the display status of the same display area may change due to the change of the myoelectric difference. A change of the display status of each display area is determined based on the changes of the plurality of myoelectric differences, so that the myoelectric animation of the virtual character is generated.

In some exemplary embodiments, the myoelectric animation of the virtual character may include a plurality of animations. For example, the myoelectric animation of the virtual character may include both a front myoelectric animation and a back myoelectric animation when there are many muscle groups mobilized during moving of the user, including both a front muscle group and a back muscle group.

As shown in FIG. 26, the process 2600 may further include:

S2660. Display the myoelectric animation of the virtual character on the user interface 380.

The motion data display system 160 may display the myoelectric animation of the virtual character on the user interface 380. The user can clearly and visually find a difference between the user and the professional by viewing the myoelectric animation of the virtual character, thereby correcting the moving motion.

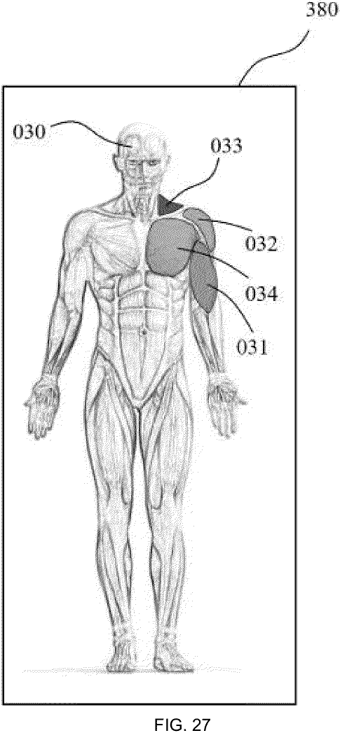

For ease of presentation, a motion of biceps lifting performed by a left arm is used as an example for description. In the motion of biceps lifting, main muscles that participate in moving include a biceps, a deltoid, a trapezius, and a pectoral muscle. FIG. 27 is a schematic diagram of the user interface 380 according to other embodiments of this disclosure. In FIG. 27, the myoelectric animation 030 of the virtual character may be displayed on the user interface 380, where the virtual user character and the virtual reference character are represented as the same virtual character. The user interface 380 shown in FIG. 27 corresponds to a myoelectric animation at a time during the motion of biceps lifting. During the motion of biceps lifting, a main force-generating point should theoretically be the biceps brachii, and in some cases, the pectoral muscle may generate force slightly, for example, when the user does not keep the chin up and the chest out. In a standard motion of biceps lifting, the trapezius should not or seldom participate in generating force. As shown in FIG. 27, the display color of the display area 033 corresponding to the trapezius in the myoelectric animation 030 of the virtual character is red, indicating that when the user performs biceps lifting, the trapezius generates relatively great force greater than the force generated by the force generated by the trapezius in the standard motion of biceps lifting.

As described above, in some exemplary embodiments, the motion data may include the plurality of pieces of posture data. Correspondingly, the reference motion data may include the plurality of pieces of reference posture data. In this case, step S2160 may include generating and displaying a posture animation of the virtual character on the user interface 380 to display the comparison between the motion data and the reference motion data. The animation of the virtual character may include the posture animation of the virtual character. In some exemplary embodiments, a moving speed and a moving angle may also affect an exercise effect. For example, in a process of biceps lifting, when a position of a lowest point is not low enough, the exercise effect is not optimal either. As another example, in a process of biceps lifting, a speed of lowering a forearm needs to be controlled, and when the speed of lowering the forearm is too high, the exercise effect is not optimal either. Therefore, it is necessary to compare the posture data with the reference posture data to improve the exercise effect.

In some exemplary embodiments, the virtual user character and the virtual reference character may be represented as two independent virtual characters. In some exemplary embodiments, the virtual user character and the virtual reference character may be represented as the same virtual character. When the motion data includes the plurality of pieces of posture data, to clearly and visually display a difference between the posture data and the reference posture data, the virtual user character and the virtual reference character may be represented as two independent virtual characters. In this case, the virtual user character and the virtual reference character may be three-dimensional animation models prepared by using three-dimensional animation software. The motion data display system 160 may control parameters of the three-dimensional animation based on the posture data to generate the posture animation. The parameters of the three-dimensional animation include but are not limited to bone displacement, bone rotation, bone scaling, and the like. A posture of the virtual user character may match the posture data. A posture of the virtual reference character may match the reference posture data.

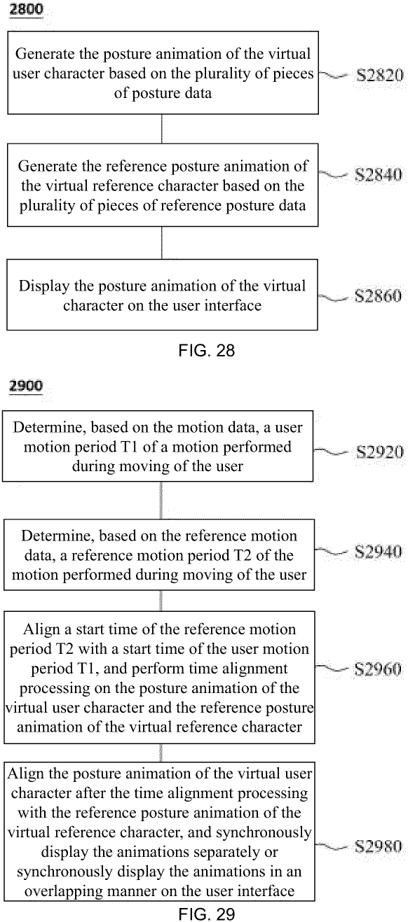

When the virtual user character and the virtual reference character may be represented as two independent virtual characters, the posture animation of the virtual character may include a posture animation of the virtual user character and a reference posture animation of the virtual reference character. FIG. 28 is an exemplary flowchart for generating a posture animation according to some exemplary embodiments of this disclosure. As shown in FIG. 28, the process 2800 may include the following steps.

S2820. Generate the posture animation of the virtual user character based on the plurality of pieces of posture data.

As described above, the virtual user character matches the plurality of pieces of posture data of the user. The motion data display system 160 may control the virtual user character based on the plurality of pieces of posture data, so that the posture of the virtual user character is consistent with the posture data during moving of the user.

S2840. Generate the reference posture animation of the virtual reference character based on the plurality of pieces of reference posture data.

As described above, the virtual reference character matches a plurality of pieces of reference data of the user. The motion data display system 160 may control the virtual reference character based on the plurality of pieces of reference posture data, so that the posture of the virtual reference character is consistent with the posture data during moving of the professional.

S2860. Display the posture animation of the virtual character on the user interface 380.

In some exemplary embodiments, displaying the posture animation of the virtual character on the user interface 380 may be synchronously displaying the posture animation of the virtual user character and the reference posture animation of the virtual reference character on the user interface 380 separately. In some exemplary embodiments, displaying the posture animation of the virtual character on the user interface 380 may be synchronously displaying the posture animation of the virtual user character and the reference posture animation of the virtual reference character in an overlapping manner on the user interface 380 directly and separately.

FIG. 29 is an exemplary flowchart for displaying a posture animation according to some exemplary embodiments of this disclosure. As shown in FIG. 29, the process 2900 may include the following steps.

S2920. Determine, based on the motion data, a user motion period T1 of a motion performed during moving of the user.

Step S2920 is basically consistent with step S2320 and is not described herein again.

S2940. Determine, based on the reference motion data, a reference motion period T2 of the motion performed during moving of the user.

Step S2940 is basically consistent with step S2340 and is not described herein again.

S2960. Align a start time of the reference motion period T2 with a start time of the user motion period T1, and perform time alignment processing on the posture animation of the virtual user character and the reference posture animation of the virtual reference character.

Specifically, the motion data display system 160 may obtain a motion start point in the user motion period T1 and a motion start point in the reference motion period T2 and align the two motion start points, thereby performing time alignment processing on the posture animation of the virtual user character and the reference posture animation of the virtual reference character, to form a comparison. Therefore, it is convenient for the user to clearly and visually observe a difference between a posture of the user and a posture of the professional and a difference between a moving speed of the user and a moving speed of the professional in a full motion period.

As shown in FIG. 29, the process 2900 may further include:

S2980. Align the posture animation of the virtual user character after the time alignment processing with the reference posture animation of the virtual reference character, and synchronously display the animations separately or synchronously display the animations in an overlapping manner on the user interface 380.

Displaying synchronously and separately may be displaying the posture animation of the virtual user character and the reference posture animation of the virtual reference character at different parts separately. For example, the posture animation of the virtual user character may be displayed in the upper half of the user interface 380 and the reference posture animation of the virtual reference character may be displayed in the lower half of the user interface 380. Alternatively, the posture animation of the virtual user character may be displayed in the left half of the user interface 380 and the reference posture animation of the virtual reference character may be displayed in the right half of the user interface 380.

Synchronously displaying in the overlapping manner may be synchronously displaying the posture animation of the virtual user character and the reference posture animation of the virtual reference character in the overlapping manner on the user interface 380. Three-dimensional models used by the virtual user character and the virtual reference character are the same. Therefore, during moving of the user, parts of the virtual user character in the same posture as the virtual reference character may overlap or basically overlap, but parts in different postures do not overlap. Therefore, overlapping of the posture animation of the virtual user character and the reference posture animation of the virtual reference character for synchronous displaying may more intuitively and clearly show the difference between the posture of the user and the posture of the professional to help the user perform an adjustment.

As described above, the motion data may include both the plurality of pieces of myoelectric data and the plurality of pieces of posture data. In this case, the animation of the virtual character may include the myoelectric animation of the virtual character and the posture animation of the virtual character. Step S2160 may include generating and displaying the myoelectric animation of the virtual character and the posture animation of the virtual character on the user interface 380 to display the comparison between the motion data and the reference motion data. In this case, the myoelectric animation of the virtual character may include the myoelectric animation of the virtual user character and the reference myoelectric animation of the virtual reference character. The posture animation of the virtual character may include the posture animation of the virtual user character and the reference posture animation of the virtual reference character.

In some exemplary embodiments, displaying the animation of the virtual character on the user interface 380 may be displaying the myoelectric animation of the virtual user character, the reference myoelectric animation of the virtual reference character, the posture animation of the virtual user character, and the reference posture animation of the virtual reference character on the user interface 380 separately. The user may simultaneously view the myoelectric animation of the virtual user character, the reference myoelectric animation of the virtual reference character, the posture animation of the virtual user character, and the reference posture animation of the virtual reference character on the user interface 380. The user may also selectively view the myoelectric animation of the virtual user character and the reference myoelectric animation of the virtual reference character, or the posture animation of the virtual user character and the reference posture animation of the virtual reference character on the user interface 380. Specifically, the user may select the type of the animation to be viewed on the user interface 380.

In some exemplary embodiments, the motion data display system 160 may alternatively combine the myoelectric animation of the virtual user character with the posture animation of the virtual user character to generate an animation of the virtual user character, and combine the posture animation of the virtual user character with the reference posture animation of the virtual reference character to generate a reference animation of the virtual reference character. The animation of the virtual user character can display both the myoelectric animation during moving of the user and the posture animation during moving of the user. The animation of the virtual reference character can display both the reference myoelectric animation during moving of the professional and the reference posture animation during moving of the professional. Displaying the animation of the virtual character on the user interface 380 may be displaying the animation of the virtual user character and the reference animation of the virtual reference character separately. Displaying the animation of the virtual character on the user interface 380 may alternatively be displaying the animation of the virtual user character and the reference animation of the virtual reference character in an overlapping manner. When the animation of the virtual user character and the reference animation of the virtual reference character are displayed in the overlapping manner, displaying the myoelectric animation may be displaying the difference between the motion data and the reference data based on the process 2600. By observing the animation of the virtual user character and the reference animation of the virtual reference character, the user can clearly and visually find a relationship between the myoelectric data and the posture data and a problem existing in the motion of the user, and perform an adjustment in time.

It should be noted that the animation of the virtual character may be displayed in real time or in non-real time when displayed on the user interface 380. Displaying in real time may be synchronizing the animation of the virtual character with the motion of the user, so that the user can view the animation of the virtual character while moving, to perform an adjustment in time. Displaying in non-real time may be that the motion data display system 160 sends the animation of the virtual character to the mobile terminal device 140 in real time. The mobile terminal device 140 may store the animation of the virtual character. When the user requests to view the animation of the virtual character through the mobile terminal device 140, the mobile terminal device 140 may display, on the user interface 380, the animation of the virtual character stored in the mobile terminal device 140, so that the user can view the animation.

In summary, the motion data display method 2100 and system 160 provided in this disclosure may combine the motion data during moving of the user and the reference motion data of the standard motion with the virtual character, to help the user clearly and visually find the problem existing in the motion of the user by comparing the motion data displayed by the virtual character with the reference motion data, thereby helping the user move scientifically.

Basic concepts have been described above. Apparently, for a person skilled in the art, the foregoing detailed disclosure is an example only, and should not be construed as a limitation on this disclosure. Although not explicitly described herein, various changes, improvements, and modifications to this disclosure may occur to a person skilled in the art. Such changes, improvements, and modifications are suggested in this disclosure. Therefore, such changes, improvements, and modifications still fall within the spirit and scope of the exemplary embodiments of this disclosure.

In addition, specific terms are used in this disclosure to describe the embodiments of this disclosure. For example, “one embodiment”, “an embodiment”, and/or “some exemplary embodiments” shall mean a feature, a structure, or a characteristic in connection with at least one embodiment of this disclosure. Therefore, it should be emphasized and noted that two or more references to “an embodiment” or “one embodiment” or “an alternative embodiment” in various places throughout this disclosure do not necessarily refer to the same embodiment. Furthermore, some of the features, structures, or characteristics in one or more embodiments of this disclosure may be combined as appropriate.

What is claimed is:

1. A motion data display method, comprising:
obtaining motion data corresponding to motion signals at a plurality of measurement positions on a body of a user;
obtaining reference motion data corresponding to the motion data;
generating a model matching the user as a virtual character; and
displaying the virtual character on a user interface to show a comparison between the motion data and the reference motion data.

2. The motion data display method according to claim 1, wherein the virtual character includes:
a virtual user character associated with the motion data; and
a virtual reference character associated with the reference motion data.

3. The motion data display method according to claim 2, wherein
the motion data includes a plurality of pieces of myoelectric data corresponding to actual myoelectric signals at a plurality of first measurement positions, among the plurality of measurement positions, on the body of the user;
the reference motion data includes a plurality of pieces of reference myoelectric data corresponding to the plurality of pieces of myoelectric data corresponding to reference myoelectric signals at the plurality of first measurement positions;
the generating of the model matching the user as the virtual character includes generating a myoelectric animation of the virtual character; and
the displaying of the virtual character on a user interface includes displaying the myoelectric animation of the virtual character on the user interface to show the comparison between the motion data and the reference motion data.

4. The motion data display method according to claim 3, wherein
the generating of the model matching the user as the virtual character and the displaying of the virtual character include:
determining display statuses of a plurality of user display areas, corresponding to the plurality of first measurement positions, on a body of the virtual user character based on the plurality of pieces of myoelectric data, and generating a myoelectric animation of the virtual user character;
determining display statuses of a plurality of reference display areas, corresponding to the plurality of first measurement positions, of the virtual reference character based on the plurality of pieces of reference myoelectric data, and generating a reference myoelectric animation of the virtual reference character; and
displaying the myoelectric animation of the virtual character, including the myoelectric animation of the virtual user character and the reference myoelectric animation of the virtual reference character, on the user interface.

5. The motion data display method according to claim 4, wherein the
determining of the display statuses of the plurality of user display areas of the virtual user character includes:
for each of the plurality of user display areas on the body of the virtual user character:
determining, based on myoelectric data at a first measurement position corresponding to the user display area, a user force contribution ratio of a muscle at the first measurement position corresponding to the user display area to a motion of the user; and
determining a display status of the user display area based on the user force contribution ratio.

6. The motion data display method according to claim 4, wherein the determining of the display statuses of the plurality of reference display areas of the virtual reference character includes:
for each of the plurality of reference display areas on a body of the virtual reference character:
determining, based on reference myoelectric data at a first measurement position corresponding to the reference display area, a reference force contribution ratio of a muscle at the first measurement position corresponding to the reference display area to the motion of the user; and
determining a display status of the reference display area based on the reference force contribution ratio.

7. The motion data display method according to claim 4, wherein
the displaying of the myoelectric animation of the virtual character on the user interface includes:
displaying the myoelectric animation of the virtual user character and the reference myoelectric animation of the virtual reference character separately on the user interface, including:
determining, based on the motion data, a user motion period of a motion of the user;
determining, based on the reference motion data, a reference motion period of the motion of the user;
performing a duration adjustment on the myoelectric animation of the virtual user character to make the user motion period following the duration adjustment consistent with the reference motion period;
aligning a start time of the reference motion period with a start time of the user motion period following the duration adjustment to perform time alignment processing on the myoelectric animation of the virtual user character following the duration adjustment and the reference myoelectric animation of the virtual reference character; and
synchronously displaying the myoelectric animation of the virtual user character and the reference myoelectric animation of the virtual reference character separately.

8. The motion data display method according to claim 3, wherein
the virtual user character and the virtual reference character are displayed as a same virtual character on the user interface, such that the plurality of display areas correspond to the plurality of first measurement positions; and
the generating of the model matching the user as the virtual character and the displaying of the virtual character include:
determining, based on the plurality of pieces of myoelectric data and the plurality of pieces of reference myoelectric data, a plurality of myoelectric differences corresponding to the plurality of first measurement positions,
determining display statuses of a plurality of display areas of the virtual character based on the plurality of myoelectric differences,
generating the myoelectric animation of the virtual character, and
displaying the myoelectric animation of the virtual character on the user interface.

9. The motion data display method according to claim 8, wherein the
determining of the plurality of myoelectric differences includes:
for each of the plurality of first measurement positions:
determining, based on myoelectric data corresponding to the first measurement position, a user force contribution ratio of a muscle corresponding to the first measurement position to a motion of the user;
determining, based on reference myoelectric data corresponding to the first measurement position, a reference force contribution ratio of the muscle corresponding to the first measurement position to the motion of the user; and
determining, based on the user force contribution ratio and the reference force contribution ratio corresponding to the first measurement position, a myoelectric difference corresponding to the first measurement position.

10. The motion data display method according to claim 2, wherein
the motion data further includes:
a plurality of pieces of posture data corresponding to actual postures at a plurality of second measurement positions on the body of the user, wherein the plurality of measurement positions includes the plurality of second measurement positions;
the reference motion data including a plurality of pieces of reference posture data corresponding to the plurality of pieces of posture data, wherein the plurality of pieces of reference posture data corresponds to reference postures at the plurality of second measurement positions; and
the generating of the model matching the user as the virtual character and the displaying of the virtual character include:
generating a posture animation of the virtual character, and
displaying the posture animation of the virtual character on the user interface to show the comparison between the motion data and the reference motion data.

11. The motion data display method according to claim 10, wherein
the generating of the model matching the user as the virtual character and the displaying of the virtual character include:
generating a posture animation of the virtual user character based on the plurality of pieces of posture data;
generating a reference posture animation of the virtual reference character based on the plurality of pieces of reference posture data; and
displaying the posture animation of the virtual character, including the posture animation of the virtual user character and the reference posture animation of the virtual reference character, on the user interface.

12. The motion data display method according to claim 11, wherein
the displaying of the posture animation of the virtual character on the user interface includes:
determining, based on the motion data, a user motion period of a motion of the user;
determining, based on the reference motion data, a reference motion period of the motion of the user;
aligning a start time of the reference motion period with a start time of the user motion period to perform time alignment processing on the posture animation of the virtual user character and the reference posture animation of the virtual reference character;
aligning the posture animation of the virtual user character following the time alignment processing with the reference posture animation of the virtual reference character; and
synchronously displaying the posture animation of the virtual user character and the reference posture animation of the virtual reference character with or without overlapping.

13. A motion data display system, comprising:
at least one storage medium storing a set of instructions for motion data display; and
at least one processor in communication with the at least one storage medium, wherein during operation, the at least one processor executes the set of instructions to:
obtain motion data corresponding to motion signals at a plurality of measurement positions on a body of a user,
obtain reference motion data corresponding to the motion data,
generat a model matching the user as a virtual character, and
display the virtual character on a user interface to show a comparison between the motion data and the reference motion data.

14. The motion data display system according to claim 13, wherein the virtual character includes:
a virtual user character associated with the motion data; and
a virtual reference character associated with the reference motion data.

15. The motion data display system according to claim 14, wherein
the motion data includes a plurality of pieces of myoelectric data corresponding to actual myoelectric signals at a plurality of first measurement positions, among the plurality of measurement positions, on the body of the user;
the reference motion data includes a plurality of pieces of reference myoelectric data corresponding to the plurality of pieces of myoelectric data corresponding to reference myoelectric signals at the plurality of first measurement positions;
the generating of the model matching the user as the virtual character includes generating a myoelectric animation of the virtual character; and
the displaying of the virtual character on a user interface includes displaying the myoelectric animation of the virtual character on the user interface to show the comparison between the motion data and the reference motion data.

16. The motion data display system according to claim 15, wherein to generate
the model matching the user as the virtual character and the displaying of the virtual character, the at least one processor executes the set of instructions to:
determine display statuses of a plurality of user display areas, corresponding to the plurality of first measurement positions, on a body of the virtual user character based on the plurality of pieces of myoelectric data, and generate a myoelectric animation of the virtual user character;
determine display statuses of a plurality of reference display areas, corresponding to the plurality of first measurement positions, of the virtual reference character based on the plurality of pieces of reference myoelectric data, and generate a reference myoelectric animation of the virtual reference character; and
display the myoelectric animation of the virtual character, including the myoelectric animation of the virtual user character and the reference myoelectric animation of the virtual reference character, on the user interface.

17. The motion data display system according to claim 16, wherein to determine
the display statuses of the plurality of user display areas of the virtual user character, the at least one processor executes the set of instructions to:
for each of the plurality of user display areas on the body of the virtual user character:
determine, based on myoelectric data at a first measurement position corresponding to the user display area, a user force contribution ratio of a muscle at the first measurement position corresponding to the user display area to a motion of the user; and
determine a display status of the user display area based on the user force contribution ratio.

18. The motion data display system according to claim 16, wherein to determine
the display statuses of the plurality of reference display areas of the virtual reference character, the at least one processor executes the set of instructions to:
for each of the plurality of reference display areas on a body of the virtual reference character:
determine, based on reference myoelectric data at a first measurement position corresponding to the reference display area, a reference force contribution ratio of a muscle at the first measurement position corresponding to the reference display area to the motion of the user; and
determine a display status of the reference display area based on the reference force contribution ratio.

19. The motion data display system according to claim 16, wherein to display
the myoelectric animation of the virtual character on the user interface, the at least one processor executes the set of instructions to:
display the myoelectric animation of the virtual user character and the reference myoelectric animation of the virtual reference character separately on the user interface, including:
determine, based on the motion data, a user motion period of a motion of the user,
determine, based on the reference motion data, a reference motion period of the motion of the user,
perform a duration adjustment on the myoelectric animation of the virtual user character to make the user motion period following the duration adjustment consistent with the reference motion period,
align a start time of the reference motion period with a start time of the user motion period following the duration adjustment to perform time alignment processing on the myoelectric animation of the virtual user character following the duration adjustment and the reference myoelectric animation of the virtual reference character, and
synchronously display the myoelectric animation of the virtual user character and the reference myoelectric animation of the virtual reference character separately.

20. The motion data display system according to claim 15, wherein
the virtual user character and the virtual reference character are displayed as a same virtual character on the user interface, such that the plurality of display areas correspond to the plurality of first measurement positions; and
to generate the model matching the user as the virtual character and the displaying of the virtual character, the at least one processor executes the set of instructions to:
determine, based on the plurality of pieces of myoelectric data and the plurality of pieces of reference myoelectric data, a plurality of myoelectric differences corresponding to the plurality of first measurement positions,
determine display statuses of a plurality of display areas of the virtual character based on the plurality of myoelectric differences,
generate the myoelectric animation of the virtual character, and
display the myoelectric animation of the virtual character on the user interface.
